(19) United States
(12) Patent Application Publication
(10) Pub. No.: US 2007/0117986 A1
(43) Pub. Date: May 24, 2007

(54) TRANS-3,5-DISUBSTITUTEDPYRROLIDINE: ORGANOCATALYST FOR anti-MANNICH REACTIONS

(76) Inventors: Fujie Tanaka, San Diego, CA (US); Carlos F. Barbas III, Solana Beach, CA (US); Haile Zhang, San Diego, CA (US)

Correspondence Address: Edward P. Gamson, Ste. 2200, 120 S. Riverside Plaza, Chicago, IL, 60606-3945, US

(21) Appl. No.: 11/560,628

(22) Filed: Nov. 16, 2006

Related U.S. Application Data
(60) Provisional application No. 60/737,663, filed on Nov. 18, 2005.
(60) Provisional application No. 60/742,780, filed on Dec. 6, 2005.
(60) Provisional application No. 60/804,507, filed on Jun. 12, 2006.

(51) Int. Cl. C07D 263/02; C07D 233/61

(52) U.S. Cl. 548/200; 548/400; 548/215; 548/335.5; 548/530

(57) ABSTRACT

A compound of Formula I is disclosed, in which R is a substituent containing a hydrogen bond-forming atom within three atoms from the ring carbon to which the substituent is bonded; X is CH₂, O, S or NR¹, wherein R¹ is a hydrocarbyl group or an amino-protecting group having one to about 18 carbon atoms; R² is hydrido or a hydrocarbyl group containing one to about twelve carbon atoms; and R³ is hydrido or methyl, but both R² and R³ are not hydrido when X is CH₂ 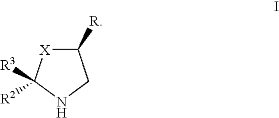
A molecule of Formula I and those in which R² and R³ can both be hydrido (Formula X) functions as a catalyst in a Mannich reaction to asymmetrically form β-aminoaldehyde or β-aminoketone diastereomeric products having two chiral centers on adjacent carbon atoms and in which the anti-diastereomers are in excess over the syn-diastereomers. Methods for carrying out those syntheses are also disclosed.

### CROSS-REFERENCE TO RELATED APPLICATION

This application claims priority from Ser. No. 60/737,663 filed on Nov. 18, 2005, Ser. No. 60/742,780 filed on Dec. 6, 2005, and Ser. No. 60/804,507 filed on Jun. 12, 2006, whose disclosures are incorporated by reference.

### TECHNICAL FIELD

The present invention contemplates an organic molecule that catalyzes anti-Mannich reactions to provide anti-products from the respective reactions with enhanced diastereo- and enantioselectivity, as well as the reactions themselves. More specifically, the present invention contemplates use of a 3-substituted-pyrrolidine to catalyze anti-Mannich reactions, and particularly wherein the 3-substituent contains a hydrogen bond-forming atom within three atoms from a ring carbon atom.

### BACKGROUND ART

Direct catalytic asymmetric Mannich reactions are highly effective carbon-carbon bond forming reactions that are used for the preparation of enantiomerically enriched amino acids, amino alcohols, and their derivatives. Because of the utility of these types of synthons, the demand for Mannich reactions that selectively afford anti- or syn-products with high enantioselectivities is high. Syn-selective direct catalytic asymmetric Mannich reactions are now common and have been performed using Zr— [Kobayashi et al., J. Am. Chem. Soc. 1998, 120, 431] Zn— [Hamada et al., J. Am. Chem. Soc. 2004, 126, 7768; Matsunaga et al., J. Am. Chem. Soc. 2004, 126, 8777; Trost et al., J. Am. Chem. Soc. 2003, 125, 338] or Cu-derived [Kobayashi et al., J. Am. Chem. Soc. 2003, 125, 2507] catalysts, Brønstead acids [Akiyama et al., Angew. Chem., Int. Ed. 2004, 43, 1566], cinchona alkaloids [Lou et al., J. Am. Chem. Soc. 2005, 127, 11256] phase-transfer catalysts [Ooi et al., Org. Lett. 2004, 6, 2397; Okada et al., Angew. Chem., Int. Ed. 2005, 44, 4564] and proline and related pyrrolidine organocatalysts [Notz et al., Adv. Synth. Catal. 2004, 346, 1131; Wang et al., Tetrahedron Lett. 2004, 45, 7243; Zhuang et al., Angew. Chem., Int. Ed. 2004, 43, 4476; Westermann et al., Angew. Chem., Int. Ed. 2005, 44, 4077; Enders et al., Angew. Chem., Int. Ed. 2005, 44, 4079; Notz et al., J. Org. Chem. 2003, 68, 9624 and references cited therein]. Methods affording syn-Mannich products have been reported for reactions involving unmodified ketones [Cobb et al., Synlett 2004, 558; Notz et al., Adv. Synth. Catal. 2004, 346, 1131 and references cited therein; Westermann et al., Angew. Chem., Int. Ed. 2005, 44, 4077; Enders et al., Angew. Chem., Int. Ed. 2005, 44, 4079; Wang et al., Tetrahedron Lett. 2004, 45, 7243; Trost et al., J. Am. Chem. Soc. 2003, 125, 338; Sugita et al., Org. Lett. 2005, 7, 5339; List, Am. Chem. Soc. 2000, 122, 9336].

Enantioselective anti-Mannich reactions are, however, considerably rarer [Kobayashi et al., J. Am. Chem. Soc. 1998, 120, 431; Hamada et al., J. Am. Chem. Soc. 2004, 126, 7768; Matsunaga et al., . Am. Chem. Soc. 2004, 126, 8777; Yoshida et al., Angew. Chem., Int. Ed. 2005, 44, 347; Mitumori et al., J. Am. Chem. Soc. 2006, 128, 1040; Kano et al., J. Am. Chem. Soc. 2005, 127, 16408; Franzen et al., J. Am. Chem. Soc. 2005, 127, 18296; Cordova et al., Tetrahedron Lett. 2002, 43, 7749]. Routes to the anti-products with high levels of diastereo- and enantioselectivities have been limited to the reactions of α-hydroxyketones using Zn catalysts [Matsunaga et al., J. Am. Chem. Soc. 2004, 126, 8777; Trost et al., J. Am. Chem. Soc. 2006, 128, 2778] and of β-ketoesters using cinchona alkaloids [Lou et al., J. Am. Chem. Soc. 2005, 127, 11256.]. Other examples of highly enantioselective anti-selective Mannich reactions of ketones use silyl enol ethers rather than unmodified ketones [Ferraris et al., J. Org. Chem. 1998, 63, 6090; Ferraris et al., J. Am. Chem. Soc. 2002, 124, 67; Hamada et al., J. Am. Chem. Soc. 2004, 126, 7768]. Even an achiral anti-selective Mannich reaction would be of interest [Takahashi et al., Chem. Lett. 2005, 34, 84]. Thus, the development of effective enantioselective anti-Mannich catalysts is a challenge in contemporary asymmetric synthesis.

### BRIEF SUMMARY OF THE INVENTION

One aspect of the present invention provides a solution to the problem of obtaining an effective enantioselective anti-Mannich reaction catalyst. That solution is a 3-substituted-pyrrolidine compound that corresponds in structure to Formula I, below, wherein the numbers within the ring structure indicate ring substituent position numbers and darkened wedge-shaped bonds indicate a bond that extends above the plane of the ring and of the page, whereas the dashed wedge-shaped bonds indicate bonds that extend below the plane of the ring and of the page, as is usual in organic chemistry. In Formula I, R (the 3-substituent) is a substituent containing a hydrogen bond-forming atom within three atoms from the ring carbon to which the substituent is bonded; X is CH₂, O, S or NR¹, wherein R¹ is a hydrocarbyl group or an amino-protecting group having one to about 18 carbon atoms; R² is hydrido or a hydrocarbyl group containing one to about twelve carbon atoms; and R³ is hydrido or methyl, but both R² and R³ are not hydrido when X is CH₂

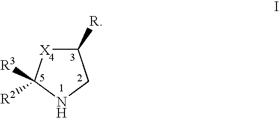

The R group in Formula I is preferably a carboxyl group, so a contemplated catalyst compound preferably corresponds in structure to Formula II, below, wherein X is CH₂, O, S or NR¹, wherein R¹ is a hydrocarbyl group or a hydrocarbyloxy group having one to about 18 carbon atoms; R² is hydrido or a hydrocarbyl group containing one to about twelve carbon atoms; and R³ is hydrido or methyl, but both R² and R³ are not hydrido when X is CH₂

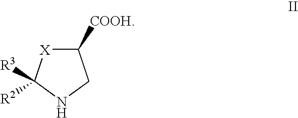

The R³ group is more preferably hydrido and a R² group is preferably other than hydrido, so a more preferred catalyst molecule corresponds in structure to Formula III, below, that is a trans-3,5-disubstitutedpyrrolidine compound wherein the 5-substituent is hydrophobic and the 3-substituent contains a hydrogen bond-forming atom within three atoms from a ring carbon atom. In a contemplated catalyst of Formula III, X is as before described, R² is a hydrocarbyl group having one to about 12 carbon atoms and R is a substituent containing a hydrogen bond-forming atom.

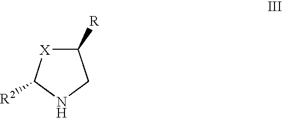

A catalyst compound in which R is a preferred carboxyl group corresponds in structure to Formula IV, below, wherein X is CH₂, O, S or NR¹, wherein R¹ is a hydrocarbyl group or a hydrocarbyloxy group having one to about 18 carbon atoms; and

R² is a hydrocarbyl group containing one to about twelve carbon atoms

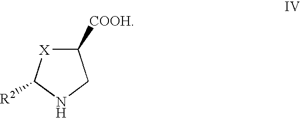

One particularly preferred catalyst is a 5-substitutedpyrrolidine-3-carboxylic acid corresponding in structure to Formula V, below, 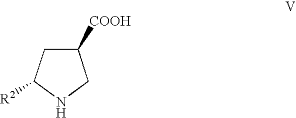
wherein R² is a hydrocarbyl group having one to about 12 carbon atoms. As is seen from structural Formulas IV and V, the substituent at the 5-position and the carboxylic acid group at the 3-position are trans to each other, or are directed below and above the plane of the five-membered ring, respectively.

A particularly preferred catalyst compound is (3R,5R)-5-methyl-3-pyrrolidinecarboxylic acid (sometimes referred to herein as RR5M3PC and as Compound 1), whose structural formula is shown below.

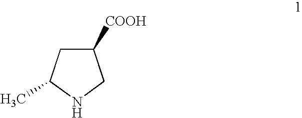

A method for asymmetrically forming β-aminoaldehyde or β-aminoketone diastereomeric products having two chiral centers on adjacent carbon atoms and in which the anti-diastereomers are in excess over the syn-diastereomers is also contemplated. That method comprises the steps of: (a) admixing an excess of an enolizable aldehyde or ketone donor molecule with an acceptor molecule having an imino group (>C═N—) that has one or no hydrogen atoms bonded to a carbon atom alpha to the carbon of the imino-unsaturation. Thus, one embodiment contemplates use of a ketone donor, whereas another embodiment contemplates use of an aldehyde donor. That admixture of donor and acceptor dissolved or dispersed in a liquid solvent in the presence of a catalyst forms an addition product reaction medium. The catalyst used corresponds in structure to a compound of Formula X, below, wherein R (the 3-substituent) is a substituent containing a hydrogen bond-forming atom within three atoms from the ring carbon to which the substituent is bonded; X is CH₂, O, S or NR¹, wherein R¹ is a hydrocarbyl group or an amino-protecting group having one to about 18 carbon atoms; R² is hydrido or a hydrocarbyl group containing one to about twelve carbon atoms; and R³ is hydrido or methyl 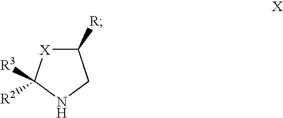
(b) The reaction medium is maintained for a time sufficient to form a β-aminoaldehyde or β-aminoketone diastereomeric products having two chiral centers on adjacent carbon atoms and in which the anti-diastereomers are in excess over the syn-diastereomers. Use of a catalyst in which R² is the hydrocarbyl group and R³ is hydrido so that R² and R are in a trans configuration provides the largest excess of anti-diastereomers over syn-diastereomers, and is preferred when the donor molecule is an aldehyde, whereas it is preferred that both R² and R³ be hydrido when the donor is a ketone. Catalysts of Formula X include those of Formulas I-V and Compound 1, each of which can be used in this method. In one preferred embodiment, the products are preferably recovered although such recovery is not required as the products can be used without further purification, as in a further synthesis.

### DETAILED DESCRIPTION OF THE INVENTION

The present invention contemplates a new compound that catalyzes an anti-Mannich reaction. That is, the compound catalyzes a Mannich reaction in which syn- and anti-diastereomers are formed, and the anti-diastereomers are formed in excess over the syn-diastereomers. This invention also contemplates a method of synthesis using that catalyst.

A contemplated catalyst is a 3-substituted-pyrrolidine compound that corresponds in structure to Formula I, below, and wherein R (the 3-substituent) is a substituent containing a hydrogen bond-forming atom within three atoms from the ring carbon to which the substituent is bonded. The hydrogen bond-forming atom is bonded directly to the pyrrolidine ring, or bonded to the ring through one or two other atoms. The ring atom, X, can be CH₂, O, S or NR¹, wherein R¹ is a hydrocarbyl group or an amino-protecting group having one to about 18 carbon atoms; R² is hydrido or a hydrocarbyl group containing one to about twelve carbon atoms; and R³ is hydrido or methyl, but both R² and R³ are not hydrido when X is CH₂

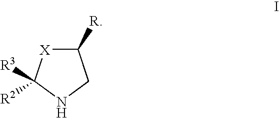

In examining structural Formula I, it is seen that four types of five-membered ring compounds are contemplated in which each has a nitrogen atom in the five-membered ring. Those four types of five-membered ring are shown below, and are a pyrrolidine (A), a thiazolidine (B), an oxazolidine (C) and an imidazolidine (D). Inasmuch as each compound contains a ring nitrogen atom and each ring contains five atoms as are present in pyrrolidine, these catalyst compounds are referred to collectively herein as pyrrolidine compounds.

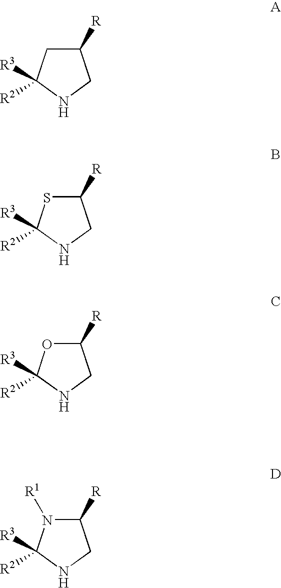

In preferred embodiments, the R group in Formula I is a carboxyl group and a preferred catalyst compound corresponds in structure to Formula II, below, wherein X is CH₂, O, S or NR¹, wherein R¹ is a hydrocarbyl group or a hydrocarbyloxy group having one to about 18 carbon atoms; R² is hydrido or a hydrocarbyl group containing one to about twelve carbon atoms; and R³ is hydrido or methyl, but both R² and R³ are not hydrido when X is CH₂

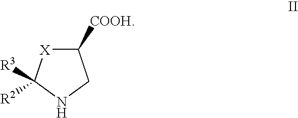

The R³ group of an above catalyst compound is more preferably hydrido and a R² group is preferably other than hydrido, so a more preferred catalyst molecule corresponds in structure to Formula III, below. Such a more preferred catalyst molecule can be referred to as a trans-3,5-disubstituted-pyrrolidine compound wherein the 5-substituent is hydrophobic and the 3-substituent contains a hydrogen bond-forming atom within three atoms from a ring carbon atom. A contemplated catalyst corresponds to Formula III, below, wherein X is as before described, R² is a hydrocarbyl group having one to about 12 carbon atoms and R is a substituent containing a hydrogen bond-forming atom.

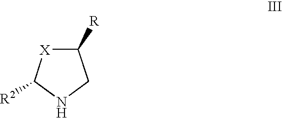

Following the before-stated preference for the R group being a carboxyl group, a still more preferred catalyst compound corresponds in structure to Formula IV, below, wherein X is CH₂, O, S or NR¹, R¹ is a hydrocarbyl group or a hydrocarbyloxy group having one to about 18 carbon atoms; and

R² is a hydrocarbyl group containing one to about twelve carbon atoms

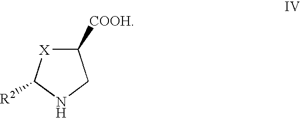

A particularly preferred catalyst is a 5-substitutedpyrrolidine-3-carboxylic acid corresponding in structure to Formula V, below, 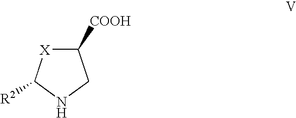
wherein R² is a hydrocarbyl group having one to about 12 carbon atoms.

As is seen from structural Formulas IV and V, the substituent at the 5-position and the carboxylic acid group at the 3-position are trans to each other, or are directed below and above the plane of the five-membered ring, respectively. It is believed that those relative positions are important to the function of the catalyst and so most of the contemplated catalysts have that trans configuration, which for most 5-substituentedpyrrolidine-3-carboxylic acids contemplated is 3R,5R. A particularly preferred catalyst compound is (3R,5R)-5-methyl-3-pyrrolidinecarboxylic acid (sometimes referred to herein as RR5M3PC and as Compound 1), whose structural formula is shown below.

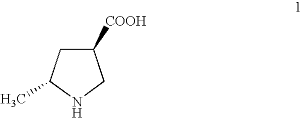

Structural formulas of illustrative catalysts are shown below, with the final compound illustrating that the hydrogen-bonding group can be a portion of a peptide having up to about ten residues.

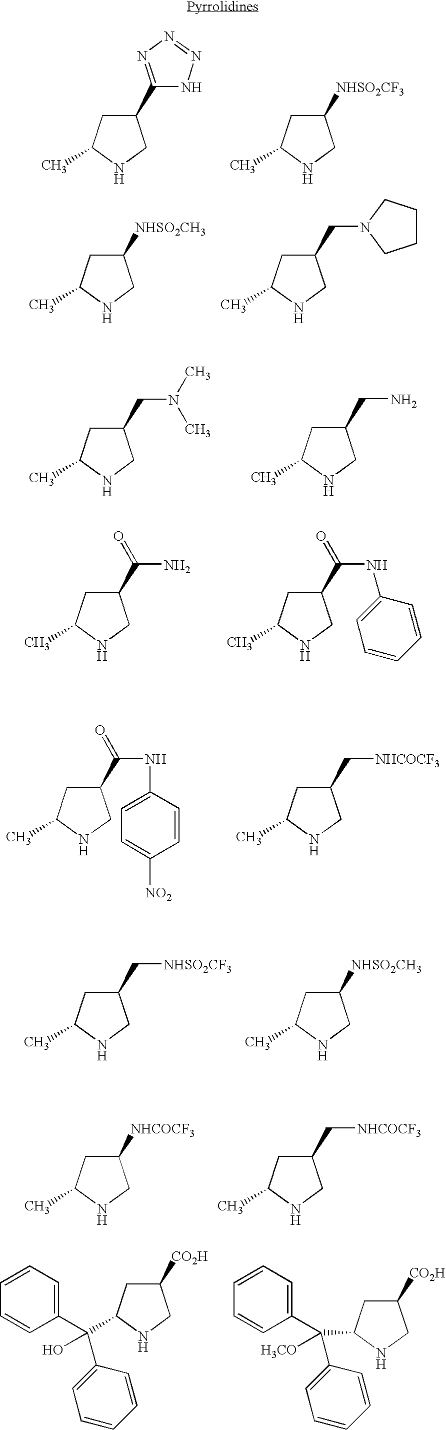

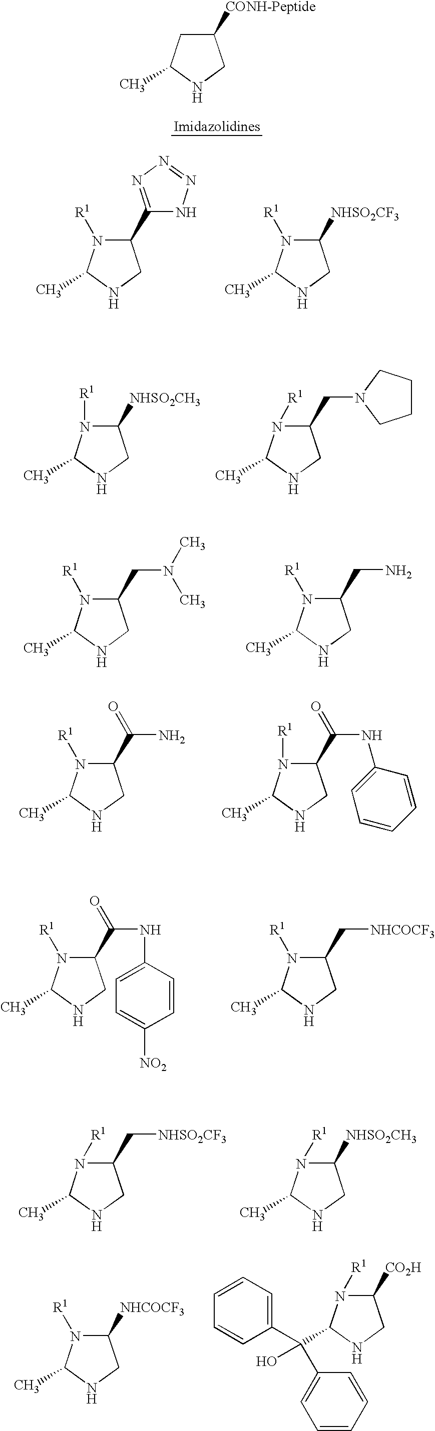

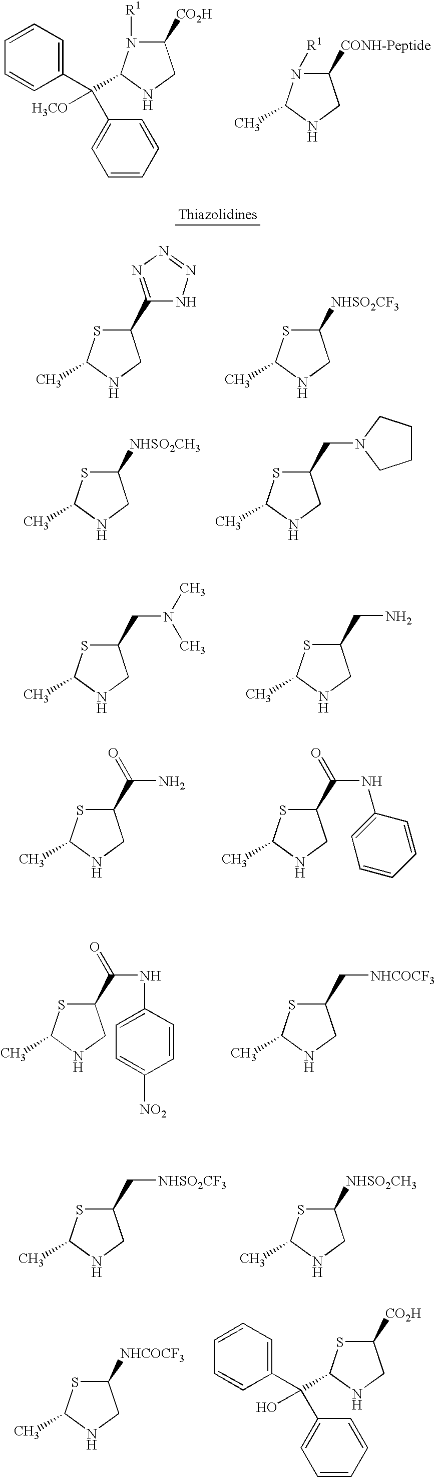

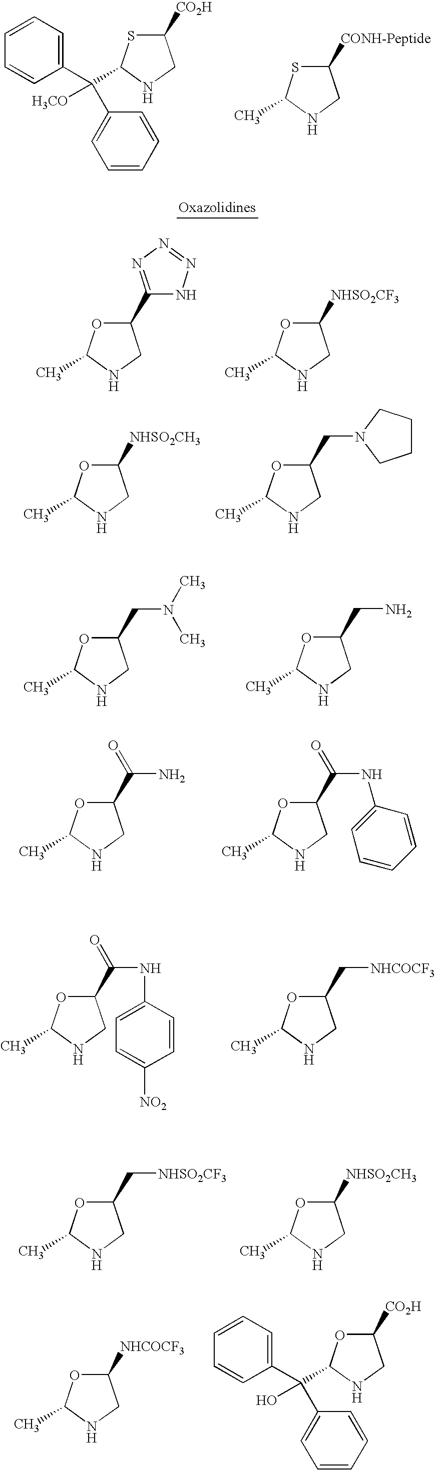

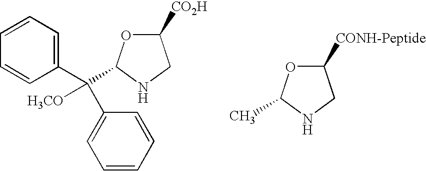

A particularly preferred hydrogen bond-forming substituent is a carboxyl group, and a particularly preferred catalyst is a 5-substitutedpyrrolidine-3-carboxylic acid corresponding in structure (and configuration) to Formula V, below, 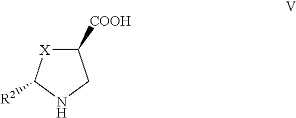
wherein R² is a hydrocarbyl group having one to about 12 carbon atoms so that the catalyst contains fewer than about 20 carbon atoms. The 3-position carboxylic acid group shown in structural Formula V is in the R configuration, and that 3-position carboxylic acid group and the substituent (R²) at the 5-position are trans to each other, or are directed above and below the plane of the five-membered ring, respectively. It is believed that those relative positions are important to the function of the catalyst and so all of the contemplated catalysts have that trans configuration, which is referred to as 3R,5R for substantially all of the 5-substituented-pyrrolidine-3-carboxylic acids contemplated.

A preferred R² group is a hydrocarbyl group having one to about 6 carbon atoms. A particularly preferred catalyst compound used illustratively herein is (3R,5R)-5-methyl-3-pyrrolidinecarboxylic acid (sometimes referred to herein as RR5M3PC and as Compound 1), whose structural formula is shown below.

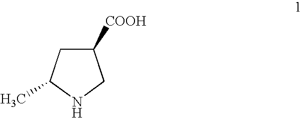

Illustrative Compound 1 is a highly diastereo- and enantioselective anti-Mannich catalyst for reactions involving unmodified aldehydes as are illustrated below in Scheme 1, wherein R is a generic organic radical and PMP is p-methoxyphenyl.

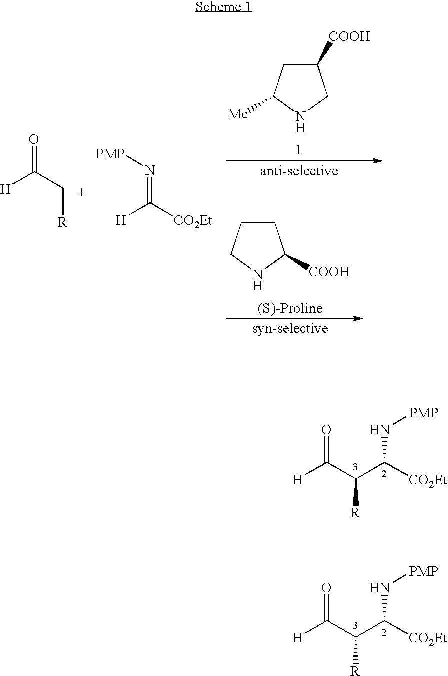

Another aspect of this invention is a method for asymmetrically forming β-aminoaldehyde or β-aminoketone diastereomeric products having two chiral centers on adjacent carbon atoms and in which the anti-diastereomers are in excess over the syn-diastereomers. That method comprises the steps of: (a) admixing an excess of a donor enolizable aldehyde or ketone molecule with an acceptor molecule having an imino group (>C═N—) that has a carbon atom bonded alpha to the carbon of the imino-unsaturation (the alpha-carbon). The alpha-carbon itself has one or no hydrogen atoms bonded to it. That admixture of donor and acceptor molecules is dissolved or dispersed in a liquid solvent in the presence of a catalyst to form an addition product reaction medium. The catalyst used corresponds in structure to a compound of Formula X, below, wherein R (the 3-substituent) is a substituent containing a hydrogen bond-forming atom within three atoms from the ring carbon to which the substituent is bonded; X is CH₂, O, S or NR¹, wherein R¹ is a hydrocarbyl group or an amino-protecting group having one to about 18 carbon atoms; R² is hydrido or a hydrocarbyl group containing one to about twelve carbon atoms; and R³ is hydrido or methyl 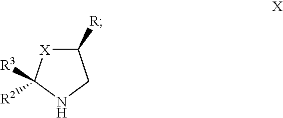
(b) The reaction medium is maintained for a time sufficient to form a β-aminoaldehyde or β-aminoketone diastereomeric product having two chiral centers on adjacent carbon atoms and in which the anti-diastereomer is in excess over the syn-diastereomer. In one preferred embodiment, the products are recovered, although such recovery is not required as the products can be used without further purification, as in a further synthesis.

As discussed in greater detail hereinbelow, catalyst compounds having R² and R³ substituents that are other than both being hydrido groups are not as useful for forming anti-compounds using ketones as donor molecules as they are for forming anti-aldehydes. Thus, preferred catalyst compounds for enantioselective anti-Mannich reaction catalysis using ketone donors have structures in which both of the R² and R³ substituents are hydrido and correspond in structure to Formula Xa, where R and X are as previously described for compounds of Formula I. Illustrative catalysts have structures that correspond to those structures shown in the Tables below.

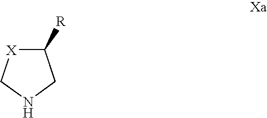

Catalysts particularly useful for anti-ketone formation have the following illustrative structures.

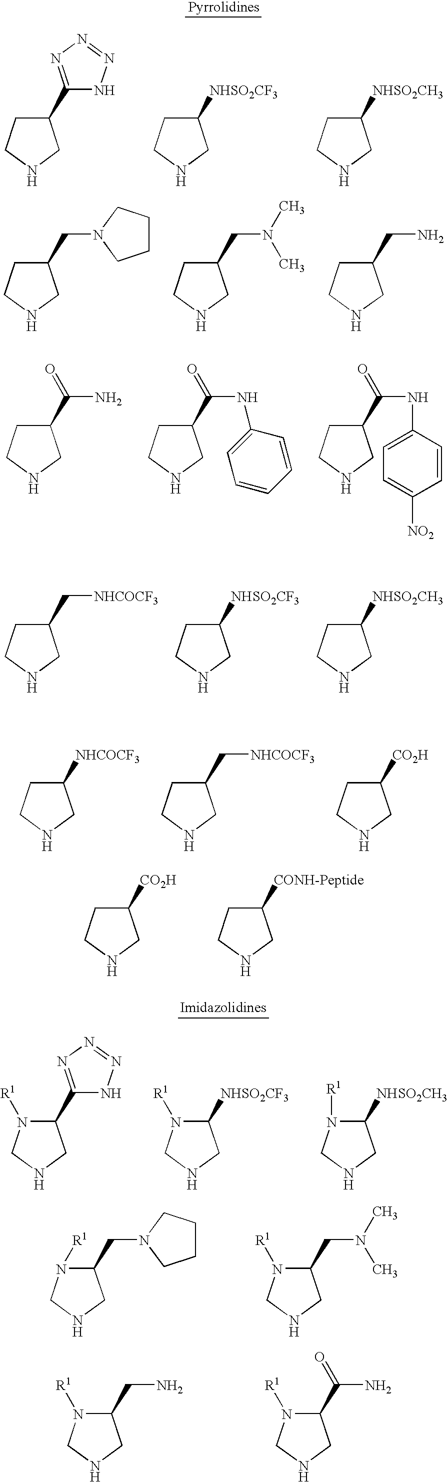

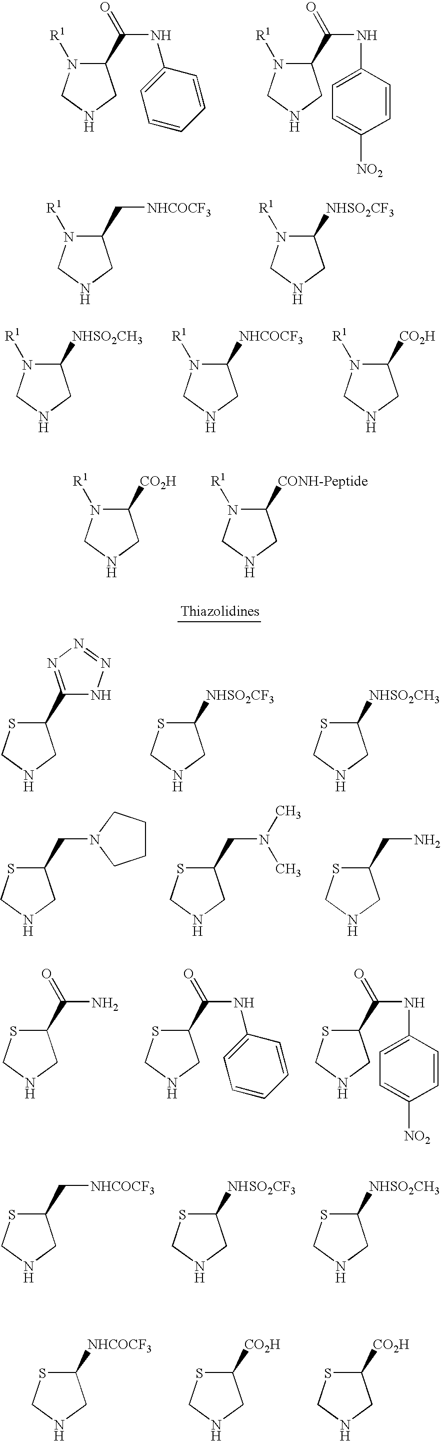

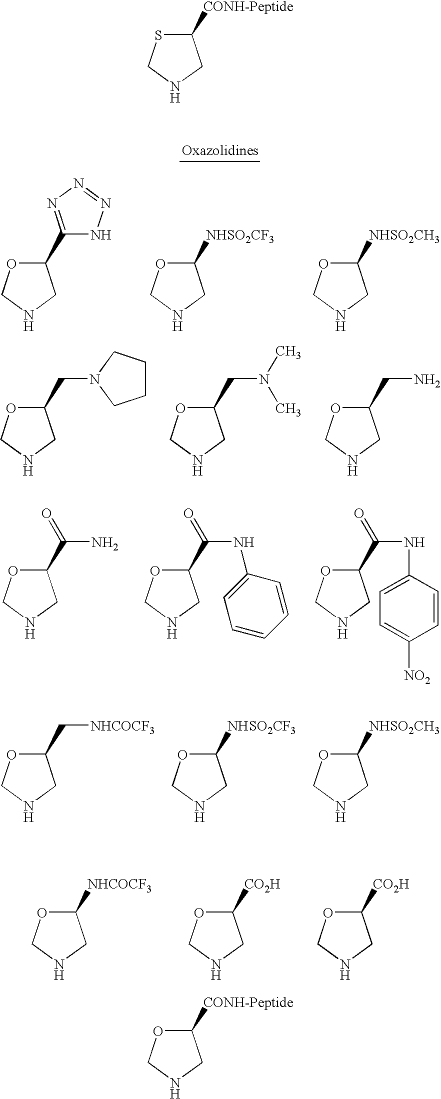

A contemplated catalyst is utilized in an amount of about 0.1 to about 50 mole percent of the amount of the acceptor aldehyde or ketone, preferably at about 0.5 to about 10 mole percent, and most preferably at about 1 to about 5 mole percent of that reagent.

In carrying out a contemplated Mannich reaction, the donor molecule contains a carbon atom that is bonded to the carbonyl carbon of the ketone or aldehyde, and that carbon atom is referred to as the alpha-carbon. The alpha-carbon also includes at least one hydrogen atom that is relatively acidic and thus can be removed to form an enolate anion at the alpha-carbon so that the donor molecule is an enolizable molecule. The alpha-carbon of the donor molecule becomes at least one chiral center in the product molecule. A donor molecule contains 2 to about 28 carbon atoms. A donor molecule more preferably contains 2 to about 10 carbon atoms.

Exemplary donor molecules contain a carbonyl group and are generally shown by the formula below

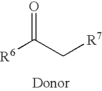

wherein R⁷ is selected from the group consisting of hydrido, C₁-C₈ straight chain, branched chain or cyclic hydrocarbyl, halogen, cyano, hydroxy, C₁-C₈-acyloxy, C₁-C₈-hydrocarbyloxy, C₁-C₈-hydrocarbylthio, azido, phthalimido and trifluoromethyl groups; and

R⁶ is selected from the group consisting of hydrido (H—), a C₁-C₁₈ straight chain, branched chain or cyclic hydrocarbyl group, an aryl group such as a phenyl, a naphthyl, pyridyl, pyrimidyl, furanyl, thiofuranyl or pyrazinyl group, or an aryl group substituted with a substituent selected from the group consisting of C₁-C₈ straight chain, branched chain or cyclic hydrocarbyl group, halogen, cyano, trifluoromethyl, nitro, hydroxyl, and a —CO₂R^{a} group, wherein R^{a} is a C₁-C₈ straight chain, branched chain or cyclic hydrocarbyl group.

Alternatively, R⁶ and R⁷ together with the depicted two carbon, two hydrogen and oxygen atoms [—C(O)—CH₂— group] form a ring structure that can contain 5 to about 9 atoms in the ring, including up to two atoms; i.e., one or two atoms, other than carbon (heteroatoms). The heteroatoms can be one or both of oxygen and sulfur.

The donor molecule ring structure so formed preferably has an even number of ring atoms, e.g., six or eight. Such a donor molecule ring compound (cyclic donor molecule) preferably has only one heteroatom present, and that that one heteroatom is preferably located symmetrically two or three carbon atoms away from the depicted carbonyl group; i.e., at the 4-position of a six-membered ring or at the 5-position of an eight-membered ring.

In another preferred embodiment, a preferred donor ring molecule contains an odd number of atoms in the ring and has two heteroatoms in the ring. Those heteroatoms are separated by a single carbon atom, and the heteroatoms are located symmetrically arrayed relative to (on each side of) the depicted carbonyl group.

In yet another embodiment, the cyclic donor molecule contains an even number of ring atoms and a protected carbonyl group located symmetrically two or three carbon atoms away from the depicted carbonyl group; i.e., at the 4-position of a six-membered ring or at the 5-position of an eight-membered ring. Illustrative protected carbonyl groups include a ketal group containing 2 to about 6 carbon atoms, an O-hydrocarbyl oxime containing 1 to about 10 carbon atoms, an N-hydrocarbyl hydrizone containing 1 to about 10 carbon atoms and a semicarbazone containing 1 to about 10 carbon atoms.

Illustrative cyclic donor molecules are illustrated below in two tables.

Cyclic Donor Molecules-I

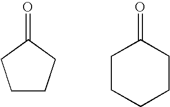

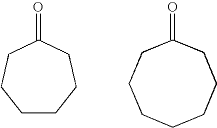

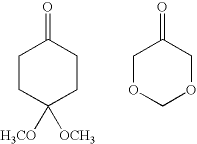

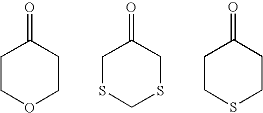

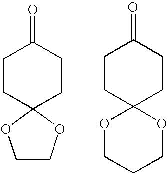

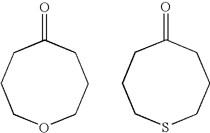

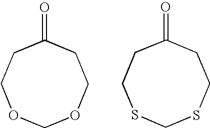

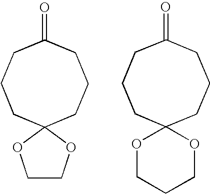

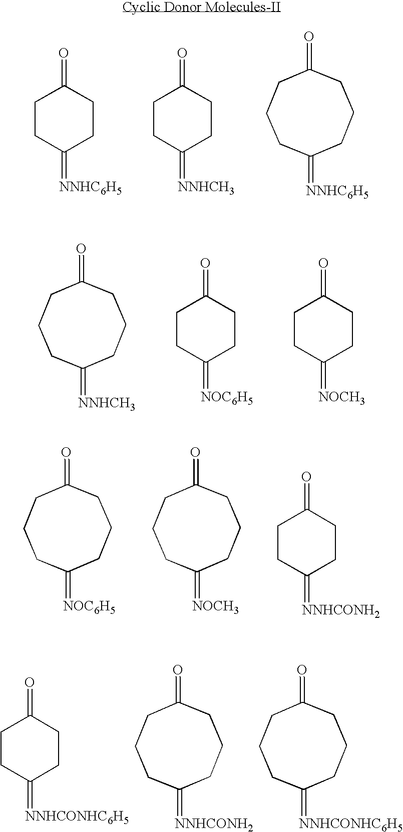

Acceptor molecules contain an imino group (>C═N—) that has one or no hydrogen atoms bonded directly to a carbon atom bonded alpha to the carbon of the imino-unsaturation. The doubly-bonded carbon atom of the imino group becomes another chiral center in the product molecule.

Illustrative acceptor compounds are shown below in Table 1, where the wavy line indicates the position of the bond between the alpha-carbon of the substituent group and the adjacent (α-) unsaturated carbon of the acceptor molecule, and NPg indicates a nitrogen atom and its protective group.

TABLE 1

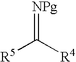

R⁴ =

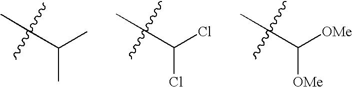

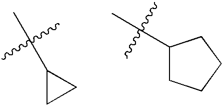

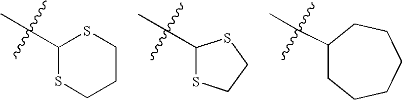

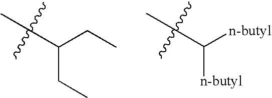

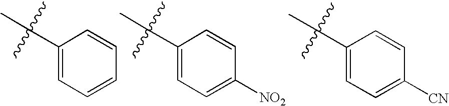

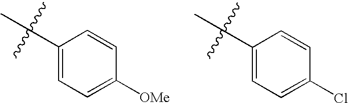

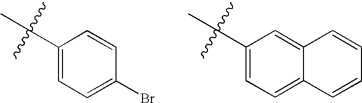

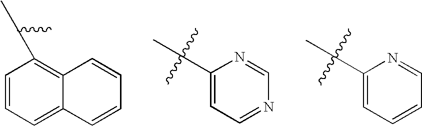

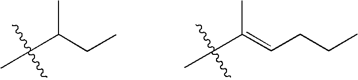

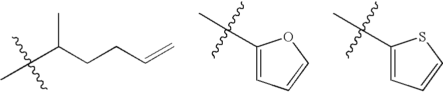

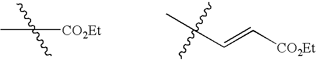

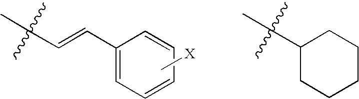

Approached differently, the acceptor molecule contains one, and preferably two, carbon atoms and can contain up to about 30 carbons, exclusive of carbon atoms present bonded to the nitrogen of the imino group; those that are part of the nitrogen atom protecting group. An acceptor more preferably contains 2 to about 12 carbons, exclusive of any carbons present in the amine protecting group that contains the substituted nitrogen atom of the imine. The R⁴ substituent can be hydrido. Alternatively, the R⁴ group can include an alpha-carbon that is bonded to one or no hydrogen atoms, and contains up to 29 carbon atoms. Such an R⁴ group comprises a substituent selected from the group consisting of:

a branched chain hydrocarbyl,

a cyclic hydrocarbyl,

a cyclic group containing 1 to 3 heteroatoms in the ring, wherein the heteroatoms are oxygen, sulfur and trisubstituted nitrogen atoms, or two of the three heteroatoms,

an aryl group such as a phenyl group, a naphthyl group, as well as a single ring or two ring heterocyclic group containing one to four heteroatoms that are oxygen, sulfur and trisubstituted nitrogen atoms such as a pyridyl, pyrimidyl, furanyl, thiofuranyl, pyrazinyl, an N-blocked imidazolyl, thiazolyl, oxazolyl, isoxazolyl, 1,2,4- or 1,2,3-triazolyl, 1,2,3- 1,2,4- 1,2,5- or 1,3,4-oxadiazolyl, 1,2,3,5-oxatriazolyl, benzofuranyl, isobenzofuranyl, thionaphthalenyl, indolyl, quinolyl, quinazolinyl, and a cinnolinyl group, wherein a third nitrogen substituent is a removable substituent as discussed previously and further including trityl groups and the like,

a sulfonylaryl group such as a —SO₂-phenyl or a —SO₂-furanyl group or other of the above aryl groups,

a nitro group,

a C₁-C₈-hydrocarbyloxycarbonyl [—C(═O)—O—C₁-C₈] group,

a substituted aryl group as discussed above wherein the substituent (—X) is selected from the group consisting of C₁-C₈ straight chain, branched chain or cyclic hydrocarbyl group, halogen, cyano, trifluoromethyl, nitro, C₁-C₈-hydrocarbyloxy and hydroxyl, and

a straight chain hydrocarbyl group substituted with 1, 2 or 3 substituents selected from the group consisting of (a) a halogen, (b) a C₁-C₈-hydrocarbyloxy group, (c) an aryl group as above, or (d) a substituted aryl group as above.

It is preferred that the alpha-carbon that is part of the R⁴ group contain no hydrogen atoms, as where R⁴ is an aryl group. If one hydrogen atom is present bonded to the alpha-carbon, the remaining R⁴ substituent is preferably bulky and contains at least four carbon atoms so that the R⁴ group can sterically hinder the approach of the amine catalyst to that alpha-carbon-bonded hydrogen. Formaldehyde is the simplest acceptor molecule and R⁴ is hydrido where formaldehyde is the acceptor.

The R⁵ group can be the same as or different from an R⁴ group. However, when R⁵ is other than hydrido, the sum of the carbon atoms in R⁴ and R⁵ can be a total of 29 atoms, the number of carbon atoms in each of R⁴ and R⁵ is adjusted accordingly so that the sum of carbon atoms in the acceptor molecule is about 30 or fewer. It is preferred that the R⁵ group be hydrido.

The word “hydrocarbyl” is used herein as a short hand term to include aliphatic as well as alicyclic groups or radicals that contain only carbon and hydrogen. Thus, alkyl, alkenyl and alkynyl groups are contemplated as are aralkyl groups such as benzyl and phenethyl, and aromatic hydrocarbons such as phenyl and naphthyl groups are also included. Where a specific hydrocarbyl substituent group is intended, that group is recited; i.e., C₁-C₄ alkyl, methyl or dodecenyl. Exemplary hydrocarbyl groups contain a chain of 1 to 18 carbon atoms, and preferably one to about 6 carbon atoms. A hydrocarbyloxy group is an ether containing a hydrocarbyl group linked to an oxygen atom. It is noted that a skilled worker would understand that an alkenyl or alkynyl substituent must have at least two carbon atoms.

The term “amino-protecting group” as used herein in relation to an R¹ group refers to one or more selectively removable substituents on the amino group commonly employed to block or protect the amino functionality. Examples of such amino-protecting groups include the formyl (“For”) group, the trityl group (Trt), the phthalimido group, the trichloroacetyl group, the chloroacetyl, bromoacetyl, and iodoacetyl groups. Urethane blocking groups, such as t-butoxycarbonyl (“Boc”), 2-(4-biphenylyl)propyl-(2)-oxycarbonyl (“Bpoc”), 2-phenylpropyl(2)oxycarbonyl (“Poc”), 2-(4-xenyl)-isopropoxycarbonyl, 1,1-diphenylethyl(1)oxycarbonyl, 1,1-diphenylpropyl(1)oxycarbonyl, 2-(3,5-dimethoxyphenyl) propyl(2)oxycarbonyl (“Ddz”), 2-(p-5-toluyl)propyl-(2)oxycarbonyl, cyclopentanyloxycarbonyl, 1-methylcyclopentanyl-oxycarbonyl, cyclohexanyloxycarbonyl, 1-methylcyclohexanyloxycarbonyl, 2-methylcyclohexanyl-oxycarbonyl, 2-(4-toluylsulfonyl)-ethoxycarbonyl, 2-(methylsulfonyl)ethoxycarbonyl, 2-(triphenyl-phosphino)ethoxycarbonyl, 9-fluoroenyl-methoxycarbonyl (“Fmoc”), 2-(trimethylsilyl)-ethoxycarbonyl, allyloxycarbonyl, 1-(trimethylsilylmethyl)prop-1-enyloxycarbonyl, 5-benzisoxalylmethoxycarbonyl, 4-acetoxybenzyl-oxycarbonyl, 2,2,2-trichloro-ethoxycarbonyl, 2-ethynyl(2)propoxycarbonyl, cyclopropyl-methoxycarbonyl, isobornyloxycarbonyl, 1-piperidyloxycarbonyl, benzyloxycarbonyl (“Z”), 4-phenylbenzyloxycarbonyl, 2-methylbenzyloxycarbonyl, α-2,4,5,-tetramethyl-benzyloxycarbonyl (“Tmz”), 4-methoxybenzyloxycarbonyl, 4-fluorobenzyl-oxycarbonyl, 4-chlorobenzyloxycarbonyl, 3-chlorobenzyloxycarbonyl, 2-chlorobenzyloxycarbonyl, dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 4-(decyloxy)-benzyloxycarbonyl, and the like, the benzoylmethylsulfonyl group, dithiasuccinoyl (“Dts’) group, the 2-(nitro)phenylsulfenyl group (“Nps’), the diphenylphosphine oxide group, and like amino-protecting groups. The species of amino-protecting group employed is usually not critical so long as the derivatized amino group is stable to the conditions of the subsequent reactions and can be removed at the appropriate point without disrupting the remainder of the compound. Preferred amino-protecting groups are Boc and Fmoc.

Further examples of amino-protecting groups embraced to by the above term are well known in organic synthesis and the peptide art and are described by, for example T. W. Greene and P. G. M. Wuts, Protective Groups in Organic Synthesis, 2^{nd} ed., John Wiley and Sons. New York., Chapter 7, 1991; M. Bodanzsky, Principles of Peptide Synthesis, 1^{st} and 2^{nd} revised eds., Springer-Verlag, New York, 1984 and 1993; and Stewart and Young, Solid Phase Peptide Synthesis, 2^{nd} ed., Pierce Chemical Co, Rockford. Ill. 1984.

A synthetic method contemplated herein is carried out in a liquid solvent, and substantially any solvent that is a liquid at a temperature of about −50° C. to about 150° C., and more preferably is liquid at a temperature of about zero ° C. to about 50° C., and most preferably is liquid at a temperature of about zero ° C. to about 40° C. Ambient room temperature (about 20-25° C.) is a particularly preferred temperature for carrying out a contemplated method.

A contemplated solvent is free of aldehydic, ketonic, acidic groups, and can dissolve or disperse the donor, acceptor and catalyst. Illustrative solvents include dimethyl sulfoxide (DMSO), dimethyl formamide (DMF), N-methyl pyrrolidinone (MNP), acetonitrile, methanol, iso-propanol, ethanol, diethyl ether, dioxane, ethyl acetate, methylene chloride, chloroform, poly(ethylene glycol) having an average molecular weight of about 200 to about 1450 and preferably about 200 to about 600, an ionic liquid, water and a combination of one of the above solvents and water.

A contemplated ionic liquid is molten at a temperature of about −50° C. to about 150° C. More preferably, a contemplated ionic liquid is liquid (molten) at or below a temperature of about 120° C. and above a temperature of minus 44° C. (−44° C.). Most preferably, a contemplated ionic liquid is liquid (molten) at a temperature of about −10° to about 100° C.

An ionic liquid is comprised of a cation and an anion. A cation of an ionic liquid is preferably cyclic and corresponds in structure to a formula selected from the group consisting of

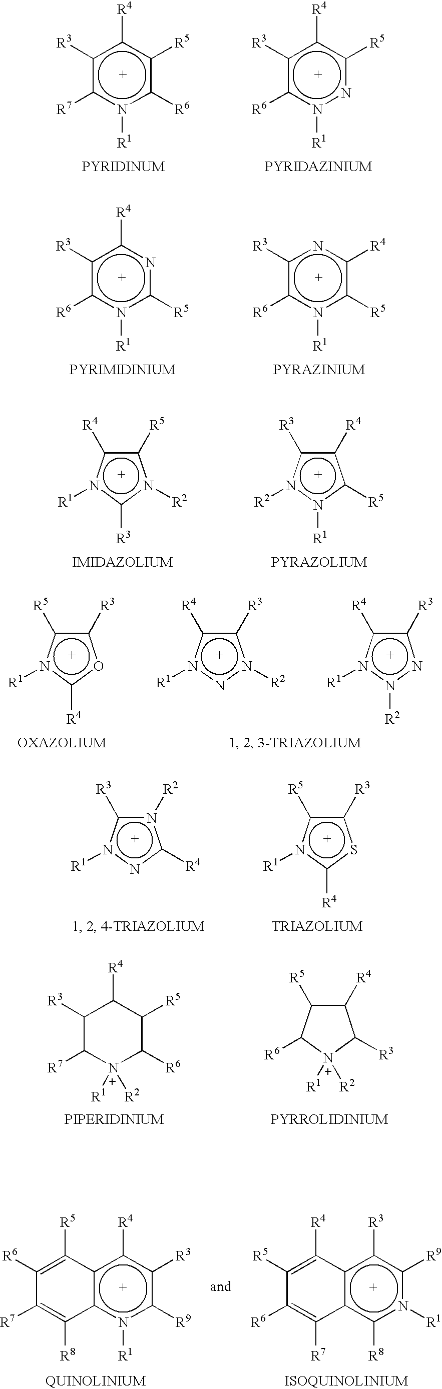

wherein R¹ and R² are independently a C₁-C₆ alkyl group or a C₁-C₆ alkoxyalkyl group, and R³, R⁴, R⁵, R⁶, R⁷, R⁸ and R⁹ (R³-R⁹), when present, are independently a hydrido, a C₁-C₆ alkyl, a C₁-C₆ alkoxyalkyl group or a C₁-C₆ alkoxy group. The “R” groups of the ionic liquids are different from those utilized with donor or acceptor molecules discussed elsewhere herein. The anions of the ionic liquid are those monovalent anions well known to those skilled in chemistry. Illustrative anions include trifluoro-methanesulfonate, trifluoroacetate, tetrafluoroborate (BF₄⁻), hexafluorophosphate (PF₆⁻), halogen, pseudohalogen, and C₁-C₆ carboxylate. Preferred anions include tetrafluoroborate and hexafluorophosphate. It is to be noted that there are two isomeric 1,2,3-triazoles. It is preferred that all R groups not required for cation formation be hydrido.

A cation that contains a single five-membered ring that is free of fusion to other ring structures is a more preferred cation. Of the more preferred cations that contain a single five-membered ring free of fusion to other ring structures, an imidazolium cation that corresponds in structure to Formula A is particularly preferred, wherein R¹, R², and R³-R⁵, are as defined before.

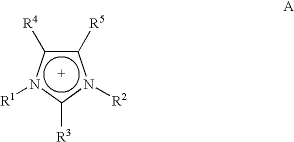

A 1,3-di-(C₁-C₆ alkyl)-substituted-imidazolium ion is a more particularly preferred cation; i.e., an imidazolium cation wherein R³-R⁵ of Formula A are each hydrido, and R¹ and R² are independently each a C₁-C₆-alkyl group or a C₁-C₆ alkoxyalkyl group. A 1-(C₁-C₆-alkyl)-3-(methyl)-imidazolium [Cₙ−mim, where n=1-6] cation is most preferred, and a tetrafluoroborate is a preferred anion.

A most preferred cation is illustrated by a compound that corresponds in structure to Formula B, below, wherein R³-R⁵ of Formula A are each hydrido and R¹ is a C₁-C₆-alkyl group or a C₁-C₆ alkoxyalkyl group.

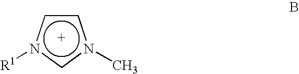

Exemplary C₁-C₆ alkyl groups and C₁-C₄ alkyl groups include methyl, ethyl, propyl, isopropyl, butyl, sec-butyl, iso-butyl, pentyl, isopentyl, hexyl, 2-ethylbutyl, 2-methylpentyl and the like. Corresponding C₁-C₆ alkoxy groups contain the above C₁-C₆ alkyl group bonded to an oxygen atom that is also bonded to the cation ring. An alkoxyalkyl group thus contains an ether group bonded to an alkyl group, and here contains a total of up to six carbon atoms.

An anion for a contemplated ionic liquid cation is preferably tetrafluoroborate or hexafluorophosphate ion, although other ions such as a trifluoromethanesulfonate or trifluoroacetate anion, as well as a halogen ion (chloride, bromide, or iodide), perchlorate, a pseudohalogen ion such as thiocyanate and cyanate or C₁-C₆ carboxylate. Pseudohalides are monovalent and have properties similar to those of halides [Schriver et al., Inorganic Chemistry, W.H. Freeman & Co., New York (1990) 406-407]. Pseudohalides include the cyanide (CN⁻¹), thiocyanate (SCN⁻¹), cyanate (OCN⁻¹), fulminate (CNO⁻¹) and azide (N₃⁻¹) anions. Carboxylate anions that contain 1-6 carbon atoms (C₁-C₆ carboxylate) are illustrated by formate, acetate, propionate, butyrate, hexanoate, maleate, fumarate, oxalate, lactate, pyruvate and the like.

The reaction medium that is formed is maintained for a time period sufficient to form a β-aminoaldehyde or β-aminoketone diastereomeric product having two chiral centers on adjacent carbon atoms and in which the anti-diastereomer is in excess over the syn-diastereomer. Typical maintenance times range from about 30 minutes to one to two days, with the time required to obtain a maximal yield being readily determined for a particular set of reaction conditions using standard assay techniques such as gas and thin layer chromatography.

In the reaction of unmodified aldehydes with N-p-methoxyphenyl-protected (PMP-protected) imines catalyzed by the natural amino acid (S)-proline, (2S,3S)-syn-amino aldehydes are obtained with high enantioselectivities [Notz et al., J. Org. Chem. 2003, 68, 9624 and references cited therein] (Scheme 1). Although reactions involving some pyrrolidine derivatives afford anti-diastereomers as their major products, the enantioselectivities obtained with these organocatalysts are moderate [Notz et al., J. Org. Chem. 2003, 68, 9624 and references cited therein].

In order to design catalysts that provide anti-products with high levels of enantioselectivities, the key factors that control the diastereo- and enantioselectivities of (S)-proline-catalyzed reactions were re-examined [Bahmanyar et al., Org. Lett. 2003, 5, 1249] (Scheme 2A, below).

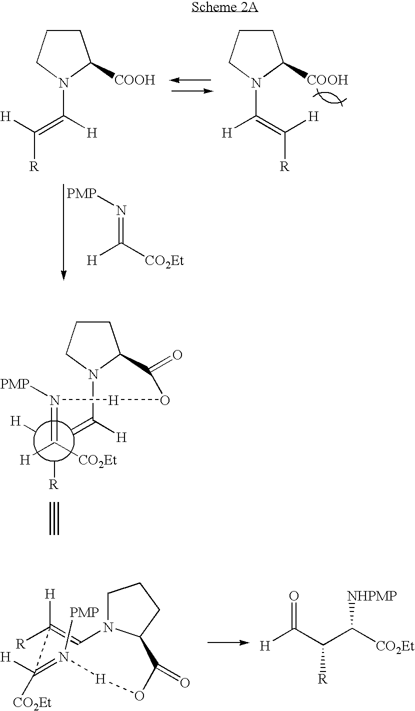

Here, four considerations are key: (1) (E)-enamine intermediates predominate due to their inherent stability and due to steric interactions with the pyrrolidine ring of the catalyst. (2) The s-trans conformation of the (E)-enamine reacts in the C—C bond-forming transition state because the s-cis conformation of the enamine results in steric interaction of the enamine with the substituent at the 2-position of the pyrrolidine ring. (3) C—C bond formation occurs at the re-face of the enamine intermediate. Reaction face selection is controlled by hydrogen bond formation between the carboxylic acid of the catalyst with the imine (or proton-transfer from the carboxylic acid to the imine nitrogen). (4) The enamine attacks the si-face of the (E)-imine wherein facial selectivity of the attack on the imine is also controlled by the hydrogen bond between the imine nitrogen and the carboxylic acid of the catalyst. The hydrogen bond also increases the electrophilicity of the imine and accelerates the reaction.

To enantioselectively form the anti-products, the reaction face of either the enamine or the imine must be opposite that utilized in the proline-catalyzed reactions. Because the carboxylic acid at the 2-position of proline impacts stereoselection in the ways described above, the steric and acidic roles of this group were deemed to need to be separated in the new catalyst. In doing so, the face selection can be modified at either the enamine or imine faces.

For example, it was thought that a pyrrolidine derivative bearing substituents at 2- and 4-positions (or at 3- and 5-positions) (Scheme 2B) would serve as an anti-Mannich catalyst. That structure was arrived at by assuming that if the steric features of the carboxylic acid at the 2-position of the proline were maintained but its acidic features removed, this substituent could be used to fix the enamine in the s-trans conformation (see point 2 above). This substituent could be a methyl group or other alkyl or aryl group that cannot engage the imine in a hydrogen bonding interaction.

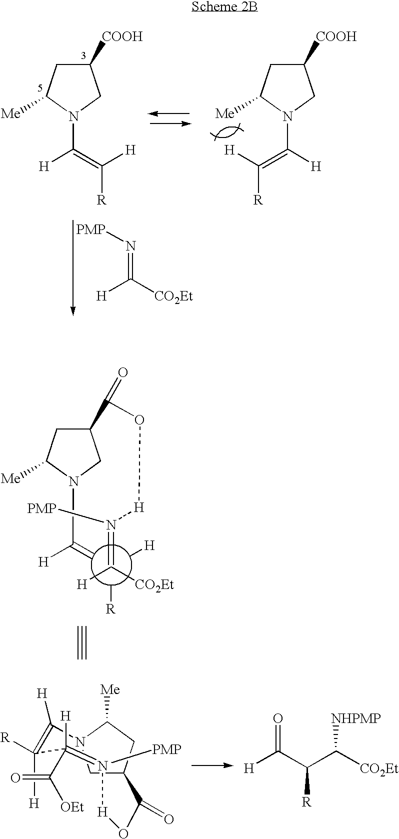

The acidic function of the carboxylic acid was then moved around the ring in order to affect control of enamine and imine face selection (see points 3 and 4). This acidic substituent could be a carboxylic acid as in proline or another acidic functional group that is able to hydrogen bond with the imine nitrogen to direct the facial selectivity of the enamine and the imine while enhancing the reactivity of the imine. In order to avoid steric interactions between the substituent at the 5-position of the new catalyst and the imine in the transition state and to enhance enamine face selectivity, the relationship between the substituents at 3- and 5-positions should be trans.

Based on these considerations, we designed a new catalyst (3R,5R)-5-methyl-3-pyrrolidine-carboxylic acid (RR5M3PC, 1). A feasible lowest energy transition state of the Mannich reaction catalyzed by 1 is represented in Scheme 2B. Computational studies of the 1-catalyzed reaction of propionaldehyde and N-PMP-protected α-imino methyl glyoxylate using HF/6-31G* level of theory [Bahmanyar et al., Org. Lett. 2003, 5, 1249] were used to test our design prior to synthesis of 1. The diastereo- and enantioselectivities were calculated and predicted an anti:syn ratio of 95:5 and ˜98% ee for the formation of the (2S,3R)-product.

RR5M3PC (1) [for racemic, cis- and trans-mixture of this compound, see: Juaristi et al., J. Org. Chem. 1991, 56, 2553] was synthesized (Scheme 4 hereinafter) and a variety of Mannich reactions involving unmodified aldehydes were studied; the results are summarized in Table 2. The typical reaction of Table 2 involved mixture of aldehyde (2 equivalents), N-PMP-protected α-imino ethyl glyoxylate (1 equivalent), and catalyst Compound 1 (0.05 equiv) in DMSO with stirring at room temperature. In accord with our design and computational test, the reactions catalyzed by Compound 1 afforded anti-amino aldehyde products with excellent diastereo- and enantioselectivities.

Significantly, reaction rates with catalyst Compound 1 were approximately 2- to 3-fold faster than the corresponding proline-catalyzed reactions that afforded syn-products when the catalyst loading was 5 mole percent. Because of the high catalytic efficiency of Compound 1, reactions catalyzed with only 1 or 2 mol percent of Compound 1 also afforded the desired product in a reasonable yield within a few hours (Table 2, entries 4 and 5). DMSO provided the best anti-selectivity and enantioselectivity of the solvents tested for the RR5M3PC-catalyzed Mannich reaction to afford anti-3. Reactions in DMF (anti:syn=97:3, 97% ee), CH₃CN (96:4, 96% ee), EtOAc (94:6, 96% ee), and dioxane (97:3, 95% ee) were as efficient with respect to reaction rate as in DMSO.

TABLE 2
RR5M3PC (1)-Catalyzed Mannich-type Reactions^{a}

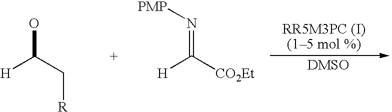

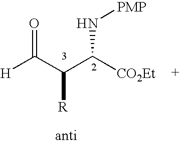

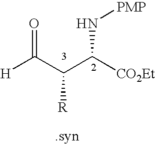

| | | time | pro- | yield | dr^{b} | ee^{c} |
|---|---|---|---|---|---|---|
| entry | R | (hours) | duct | (%) | anti:syn | (%) |
| 1 | Me | 1 | 2 | 70 | 94:6 | >99^{d} |
| 2 | i-Pr | 3 | 3 | 85 | 98:2 | 99 |
| 3 | n-Bu | 0.5 | 4 | 54 | 97:3 | 99 |
| 4^{e,f} | n-Bu | 1 | 4 | 71 | 97:3 | 99 |
| 5^{e,g} | n-Bu | 2 | 4 | 57 | 97:3 | >99 |
| 6 | n-Pent | 3 | 5 | 80 | 97:3 | >99 |
| 7^{h} | CH₂CH═CH₂ | 3 | 6 | 72 | 96:4 | >97 |

^{a}Typical conditions: To a solution of N-PMP-protected α-imino ethyl glyoxylate (0.25 mmol) and aldehyde (0.5 mmol) in anhydrous DMSO (2.5 mL), catalyst RR5M3PC (1) (0.0125 mmol, 5 mol % to the imine) was added and the mixture was stirred at room temperature.
^{b}The diastereomeric ratio (dr) was determined by ¹H NNR.
^{c}The ee of the anti-product was determined by chiral-phase HPLC analysis.
^{d}The ee was determined by HPLC analysis of the corresponding oxime prepared with O-benzyl-hydroxylamine.
^{e}The reaction was performed in a doubled scale.
^{f}Catalyst 1 (2 mol %) was used.
^{g}Catalyst 1 (1 mol %) was used.
^{h}The reaction was performed with doubled concentration for each reactant and catalyst 1.

When the reaction of entry 2 was carried out using (S)-pyrrolidinecarboxylic acid (Formula I, where R=CO₂H; R²=R³=H; X=CH₂) as the catalyst to provide Product 3, (2R,3S)-anti-3 was formed at an anti:syn ratio of 95:5, with an ee of 93%. This result suggests that the 3-carboxylic acid stereochemistry is more important for selectivity than is the 5-substituent.

A reaction similar to entry 2 above was run using two other, illustrative catalysts, as shown below in which the catalysis reactions were carried out in the presence and absence of 5-methyltetrazole as an additive. As is seen, overall yields were somewhat lower, but the anti:syn ratio and ee percentage were excellent.

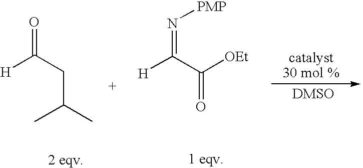

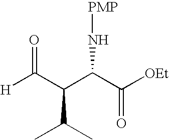

| | addi- | yield | | | |
|---|---|---|---|---|---|
| catalyst | tive | % | time | anti:syn | ee % |
| 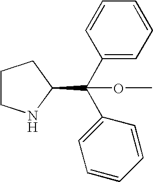 | no yes* | 61 32 | 3 days 4 hours | 9:1 9:1 | 60/56 92/54 |
| 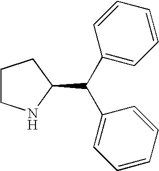 | no yes* | 20 17 | 3 days 4 hours | 9:1 13:1 | 90/8 94/— |

*5-methyltetrazole

Imidazole isomerization [Ward et al., J. Org. Chem. 2004, 69, 4808] of the anti-3 product obtained from the RR5M3PC-catalyzed reaction and of the (2S,3S)-syn-3 product obtained from the (S)-proline-catalyzed reaction [Notz et al., J. Org. Chem. 2003, 68, 9624 and references cited therein] confirmed that the major anti-product generated from the RR5M3PC-catalyzed reaction had a (2S,3R) configuration (Scheme 3).

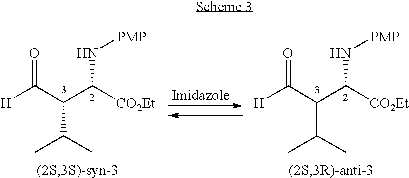

An efficient organocatalyst, RR5M3PC (Compound 1), has been developed for anti-Mannich-type reactions. This catalyst is useful for the synthesis of amino acid derivatives with excellent anti-selectivities and enantioselectivities under mild conditions.

Mannich-type reactions between unmodified ketones and N-p-methoxyphenyl (PMP)-protected α-imino esters that afford anti-products with high diastereo- and enantioselectivities, using β-proline or 3-pyrrolidinecarboxylic acid (16) as catalyst are illustrated hereinafter. The Compound (R)-16- and (S)-16-catalyzed anti-selective Mannich-type reactions of unmodified ketones afford high diastereo- and enantioselectivities. The results discussed below demonstrate that the position of the acid group on the pyrrolidine ring directs the stereoselection of the catalyzed reaction, providing either syn- or anti-Mannich products.

The before-described anti-Mannich catalyst, (3R,5R)-5-methyl-3-pyrrolidinecarboxylic acid (1) illustrates highly diastereo- and enantioselective anti-Mannich-type reactions of aldehydes using this catalyst. As noted in Scheme 2B, a key for the formation of anti-Mannich products is the use of enamine conformation below over that shown above in the C—C bond-forming transition state. Catalyst Compound 1, however, was ineffective in the Mannich-type reactions of ketones. The Compound 1-catalyzed Mannich-type reaction between 3-pentanone and N-PMP-α-imino ethyl glyoxylate was very slow (Table 3, entry 1).

Upon consideration of the transition states of the ketone reaction, we reasoned that the low efficiency of catalyst Compound 1 in the ketone reaction originated from relatively slow formation of the enamine intermediates due to steric interaction with the methyl group of the catalyst. Note that proline catalyzes the syn-Mannich-type reactions of both aldehydes [Notz et al., J. Org. Chem. 2003, 68, 9624 and references cited therein.] and ketones [Notz et al., Adv. Synth. Catal. 2004, 346, 1131 and references cited therein; Westermann et al., Angew. Chem., Int. Ed. 2005, 44, 4077; Enders et al., Angew. Chem., Int. Ed. 2005, 44, 4079].

In the case of the Mannich-type reactions of isovaleraldehyde, although both the 3-carboxylic acid and 5-methyl groups of catalyst Compound 1 were critical for excellent anti-selectivity and enantioselectivity, the 3-carboxylic acid group alone had a significant role in the stereoselection [Mitumori et al., J. Am. Chem. Soc. 2006, 128, 1040]. We reasoned that unmethylated catalyst (R)-3-pyrrolidinecarboxylic acid [(R)-16] should afford anti-Mannich products in the ketone reactions. When proton transfer occurs from the acid at the 3-position of the catalyst to the imine nitrogen, the nucleophilic carbon of enamine should be properly positioned to react with the imine, whereas the nucleophilic carbon of enamine in a different conformation should be too far from the imine carbon to form a bond.

Because Compound 16 does not have an α-substituent on the pyrrolidine, neither enamine conformation has a disfavored steric interaction and enamine formation of ketones with Compound 16 should be faster than that with Compound 1.

In fact, the Compound (R)-16-catalyzed reaction was significantly faster than the Compound 1-catalyzed reaction and afforded the anti-Mannich product (2S,3R)-18 in good yield with high diastereo- and enantioselectivities (Table 4, entry 2), supporting our design considerations. When the position of the carboxylic acid group on the pyrrolidine ring was changed from the 2- to the 3-position (that is, from proline to catalyst Compound 16), the stereochemistry of the product of the catalyzed reaction was altered from syn to anti. Catalyst Compound (S)-17, which possesses a hydrogen bond-forming atom in the sulfonamide group, also catalyzed the reaction and afforded the anti-product, but the reaction catalyzed by Compound 16 was faster and afforded higher enantioselectivity than the Compound 17-catalyzed reaction. These results indicate that the acid functionality at the 3-position on the pyrrolidine ring plays an important role in properly positioning the imine, for a faster reaction rate and for affording the anti-products with high diastereo- and enantioselectivities.

TABLE 3
Evaluation of Catalysts for the anti-Selective
Mannich-Type Reaction of 3-Pentanone^{a}

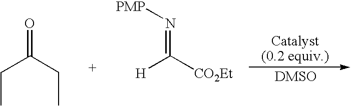

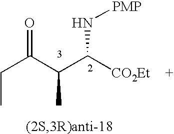

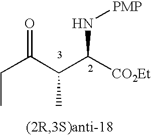

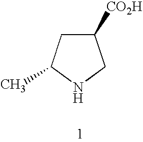

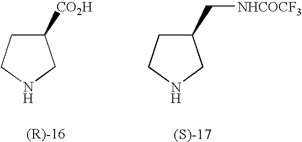

| | | | | dr^{c} | | |
|---|---|---|---|---|---|---|
| | | | yield^{b} | anti: | major | ee^{d} |
| entry | catalyst | time | (%) | syn | anti-4 | (%) |
| 1 | 1 | 3 d | <10 | — | — | — |
| 2 | (R)-16 | 29 h | 75 | 94:6 | (2S,3R) | 97 |
| 3 | (S)-17 | 3 d | 83 | 94:6 | (2R,3S) | 85 |

^{a}To a solution of N-PMP-protected α-imino ester (0.5 mmol, 1 equiv) and 3-pentanone (2.0 mL, 19 mmol, 38 equiv) in anhydrous DMSO (3.0 mL), catalyst (0.1 mmol, 0.2 equiv, 20 mol % to the imine) was added and the mixture was stirred at room temperature (25° C.).
^{b}Isolated yield (containing anti- and syn-diastereomers).
^{c}Determined by HPLC before purification.
^{d}Determined by chiral-phase HPLC for anti-4.

Evaluation of the Compound (R)-16-catalyzed reaction to afford Compound (2S,3R)-anti-18 in various solvents at room temperature showed that the reaction in 2-PrOH provided the highest reaction rate, yield, anti-selectivity, and enantioselectivity of the solvents tested (Table 4, entry 1 and Supporting Information).

TABLE 4
(R)-16-Catalyzed anti-Mannich-Type
Reactions of Ketones^{a}

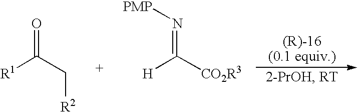

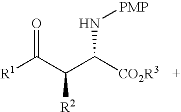

| | | | | time | | yield^{b} | dr^{c} | ee^{d} |
|---|---|---|---|---|---|---|---|---|
| entry | R¹ | R² | R³ | (h) | product | (%) | anti:syn | (%) |
| 1 | Et | Me | Et | 20 | 18 | 91 | 97:3 | 97 |
| 2^{e} | Et | Me | Et | 48 | 18 | 77 | 97:3 | 98 |
| 3 | Et | Me | t-Bu | 20 | 19 | 93 | >99:1 | 95 |
| 4 | n-Pr | Et | Et | 96 | 20 | 76 | >99:1 | 82 |
| 5 | Me | Me | Et | 5 | 21 | 85^{f} | ˜10:1 (>99:1)^{g} | 90 (>99)^{g} |
| 6^{h} | Me | Me | Et | 5 | ent-21 | 81^{f} | ˜10:1 (>99:1)^{g} | 88 (99)^{g} |
| 7 | Me | Et | Et | 10 | 22 | 81^{f} | ˜10:1 | 92 |
| 8 | Me | CH₂CH═CH₂ | Et | 14 | 23 | 85 | >95:5 | 91 |
| 9 | Me | (CH₂)₃Cl | Et | 14 | 24 | 68 | >95:5 | 84 |

^{a}Typical conditions: To a solution of imine (0.5 mmol, 1 equiv.) and ketone (5.0 mmol, 10 equiv.) in 2-PrOH (1.0 mL), Compound (R)-16 (0.05 mmol, 0.1 equiv.) was added and the mixture was stirred at 25° C.
^{b}Isolated yield (containing anti- and syn-diastereomers).
^{c}Determined by NMR of isolated products.
^{d}Determined by chiral-phase HPLC for the anti-product.
^{e}Ketone (4 equiv.), Compound (R)-16 (0.05 equiv.), at 4° C.
^{f}Containing regioisomer (˜5-10%).
^{g}Data after crystallization are shown in parentheses. The dr was determined by HPLC.
^{h}Catalyst (S)-16 was used.

Amino acid Compound (R)-16 catalyzed Mannich-type reactions between a variety of ketones and α-imino esters and afforded the anti-products in good yields with high diastereo- and enantioselectivities in most cases (Tables 4 and 5). For the reactions of unsymmetrical methyl alkyl ketones, the reaction occurred predominantly at the more substituted α-position of the ketones (Table 4, entries 5-9). The regio-, diastereo-, and/or enantiomeric purities of the anti-products were readily improved by crystallization (Table 4, entries 5, 6, Table 5, entry 3). For the reactions of 6-membered cyclic ketones, use of only 5 mol % of catalyst Compound 16 and 2 equivalents of ketone to the imine afforded the desired anti-products in good yields within approximately 12 hours.

TABLE 5
(R)-16-Catalyzed anti-Mannich-Type
Reactions of Cyclic Ketones^{a}

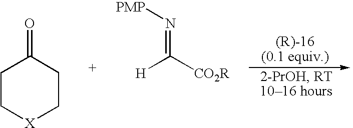

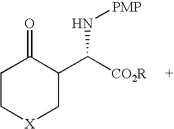

| | | | Catalyst | | yield^{b} | dr^{c} | ee^{d} |
|---|---|---|---|---|---|---|---|
| entry | X | R | (equiv) | product | (%) | anti:syn | (%) |
| 1^{c} | CH₂ | Et | 0.1 | 25 | 96 | >99:1 | 96 |
| 2 | CH₂ | i-Pr | 0.05 | 26 | 94 | >99:1 | 94 |
| 3 | CH₂ | t-Bu | 0.05 | 27 | 92 | >99:1 | 95 (99)^{f} |
| 4 | CH₂ | CH₂CH═CH | 0.05 | 28 | 95 | >99:1 | 95 |
| 5 | S | Et | 0.1 | 29 | 78 | >99:1 | 99 |
| 6 | S | Et | 0.05 | 29 | 71 | >99:1 | 97 |
| 7 | O | Et | 0.1 | 30 | 82 | >95:5 | 86 |
| 8 | C(OCH₂)₂ | Et | 0.1 | 31 | 87 | >99:1 | 97 |
| 9 | O(OCH₂)₂ | Et | 0.05 | 31 | 80 | >99:1 | 96 |

^{a}Typical conditions: Imine (0.5 mmol, 1 equiv.), ketone (1.0 mmol, 2 equiv.), Compound (R)-15 (0.05 mmol, 0.1 eguiv. or 0.025 mmol, 0.05 equiv.), 2-PrOH (1.0 mL), 25° C.
^{b}Isolated yield.
^{c}Determined by NMR of isolated products.
^{d}Determined by chiral-phase HPLC of the anti-product.
^{e}Ketone (5.0 mmol, 10 equiv) was used.
^{f}Data after crystallization.

TABLE 6
(R)-16-Catalyzed anti-Mannich-Type
Reactions of Aldehydes^{a}

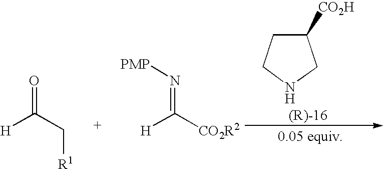

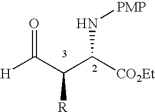

| | | | Time | Yield | dr^{b} | ee^{c} |
|---|---|---|---|---|---|---|
| entry | R¹ | R2 | (h) | (%) | anti:syn | (%) |
| 1 | Me | Et | | 75 | 93:7 | 96 |
| 2 | i-Pr | Et | 4 | 81 | 99:1 | 94 |
| 3 | n-Bu | Et | 2 | 60 | 99:1 | 95 |
| 4 | n-Pent | Et | 3 | 80 | 99:1 | >97 |
| 5 | CH₂CH═CH₂ | El | 3 | — | 99:1 | >97 |
| 6 | CH₂CH═CH(CH₂)₄CH₃ | Et | 3 | 83 | 98:2 | 99 |
| 7 | i-Pr | i-Pr | 3 | 82 | 98:2 | 91 |
| 8 | i-Pr | t-Bu | 2.5 | 82 | 99:1 | 94 |
| 9 | i-Pr | CH₂CH═CH₂ | 3 | 85 | 98:2 | 95 |

^{a}Typical reaction conditions: To a solution of N-PMP-protected α-imino ester (0.25 mmol, 1 equiv.) and aldehyde (0.5 mmol, 2 equiv) in anhydrous DMSO (2.5 mL), catalyst 1 (0.0125 mmol, 0.05 equiv, 5 mol % to the imine) was added and the mixture was stirred at room temperature.
^{b}The diastereomeric ratio (dr) was determined by ¹H NNR.
^{c}The ee of anti-product was determined by chiral-phase HPLC analysis.

Without further elaboration, it is believed that one skilled in the art can, using the preceding description, utilize the present invention to its fullest extent. The following preferred specific embodiments are, therefore, to be construed as merely illustrative, and not limiting of the remainder of the disclosure in any way whatsoever.

General Procedures

Moisture-sensitive reactions were carried out under an argon atmosphere. For thin layer chromatography (TLC), silica gel plates VWR GL60 F254 were used and compounds were visualized by irradiation with UV light and/or by treatment with a solution of phosphomolybdic acid (25 g), Ce(SO₄)₂.H₂O (10 g), and conc. H₂SO₄ (60 mL) in H₂O (940 mL) followed by heating or by treatment with a solution of p-anisaldehyde (23 mL), conc. H₂SO₄ (35 mL), and acetic acid (10 mL) in ethanol (900 mL) followed by heating. Flash column chromatography was performed using Bodman silica gel 32-63, 60 Å.

¹H NMR and ¹³C NMR spectra were recorded on INOVA-400 or Mer-300. Proton chemical shifts are given in δ relative to tetramethylsilane (δ 0.00 ppm) in CDCl₃ or to the residual proton signals of the deuterated solvent in CD₃OD (δ 3.35 ppm). Carbon chemical shifts were internally referenced to the deuterated solvent signals in CD₃Cl (δ 77.00 ppm) or CD₃OD (δ 49.00 ppm). High-resolution mass spectra were recorded on an Agilent ESI-TOF mass spectrometer. Enantiomeric excesses were determined by chiral-phase HPLC using a Hitachi instrument. Optical rotations were measured on a Perkin-Elmer 241 polarimeter.

Solvent Screen

Solvent Screen on the Mannich-Type Reaction Between 3-Pentanone and N-PMP-Protected α-Imino Ethyl Glyoxylate Using (R)-3-Pyrrolidinecarboxylic Acid^{a}

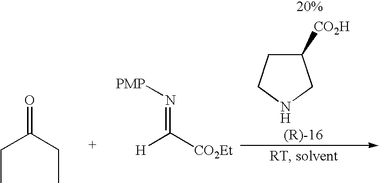

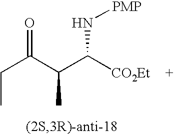

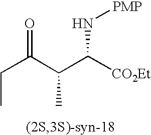

| | | | | dr^{c} | |
|---|---|---|---|---|---|
| entry | solvent | time | yield^{b} (%) | anti:syn | ee^{c} (%) |
| 1 | DMSO | 29 h | 75 | 94:6 | 97 |
| 2 | DMF | 38 h | 74 | 87:13 | 97 |
| 3 | N-Methyl-pyrrolidone | 40 h | 65 | 93:7 | 96 |
| | (NMP) | | | | |
| 4 | CHCl₃ | 3 d | 46 | 95:5 | 97 |
| 5 | CH₃CN | 3 d | 51 | 97:3 | 95 |
| 6 | Dioxane | 3 d | 30 | 74:26 | 71 |
| 7 | THF | 3 d | <10 | ND | ND |
| 8 | AcOEt | 3 d | <10 | ND | ND |
| 9 | 2-PrOH | 18 h | 90 | 97:3 | 98 |
| 10 | EtOH | 18 h | 91 | 95:5 | 92 |
| 11 | MeOH | 32 h | 79 | 96:4 | 87 |
| ^{a}Conditions: (E)-Ethyl 2-(p-methoxyphenylimino)-acetate (0.1 mmol, 1 equiv.) was dissolved in a solvent (2.5 mL) and 3-pentanone (0.4 mL, 3.8 mmol, 38 equiv) was added, followed by (R)-3-pyrrolidinecarboxylic acid (R)-16 (0.02 mmol, 0.2 equiv, 20 mol % to the imine) at room temperature (25° C.). After stirring for the indicated time at the same temperature, the reaction mixture was worked up by addition of aqueous saturated ammonium | | | | | |
| # chloride solution and was extracted with AcOEt (three or four times). The combined organic layers were dried over anhydrous MgSO₄, filtered, concentrated in vacuo, and purified by flash column chromatography (AcOEt/hexane = 1:10). For entries 9-10, the reaction mixture was concentrated in vacuo without work-up, and purified by flash column chromatography. The anti- and syn-diastereomers were not discriminated each other on TLC. | | | | | |

^{b}Isolated yield (containing anti- and syn-diastereomers).
^{c}The dr and ee of the isolated products were determined by chiral-phase HPLC using Daicel Chiralpak AS.

2-PrOH was the best solvent tested in terms of reaction rate, yield, less byproduct formation, anti-selectivity, and enantioselectivity. Although the reactions in DMSO and in 2-PrOH afforded similar diastero- and enantioselectivities, the reaction rate in 2-PrOH was approximately 2-fold faster than that in DMSO and the reaction in 2-PrOH was cleaner (less byproduct formation) than that in DMSO. An approximate order of the reaction rate of the product formation (from the solvent for faster reaction): EtOH, 2-PrOH>>DMSO, DMF, NMP, MeOH>>CH₃CN>CHCl₃>Dioxane, THF, AcOEt.
Synthesis of Catalyst Compound 1 (Scheme 4)

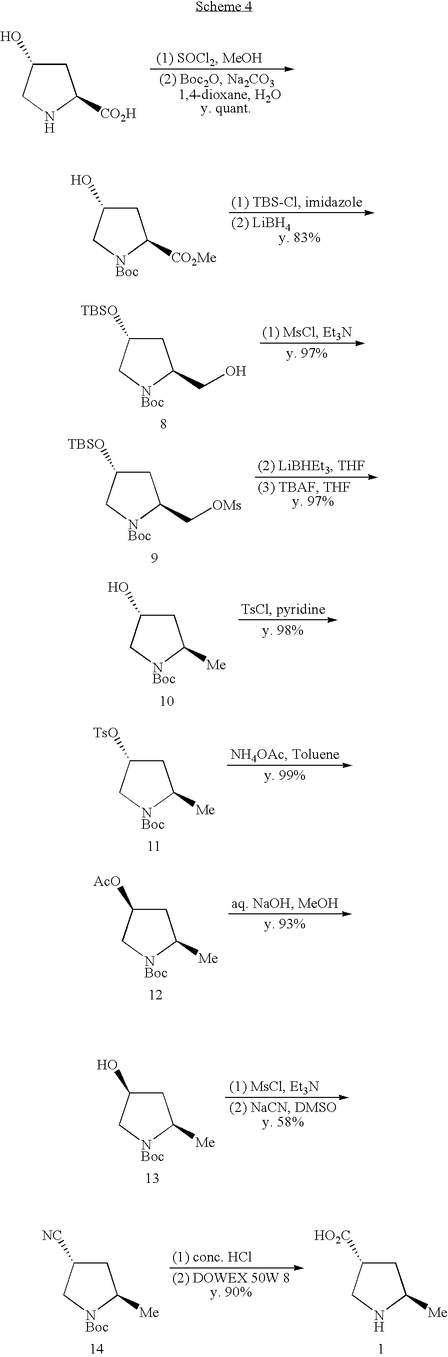

### EXAMPLE 1

### (2S,4R)-tert-butyl 4-(tert-butyldimethylsilyloxy)-2-(hydroxymethyl)-pyrrolidine-1-carboxylate (8)

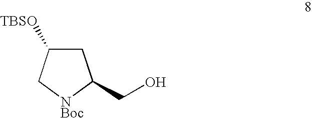

Compound 8 was synthesized from trans-4-hydroxy-L-proline by the reported procedures [Rosen et al., J. Med. Chem. 1988, 31, 1598]. ¹H NMR (400 MHz, CDCl₃): δ 0.08 (s, 6H), 0.87 (s, 9H), 1.47 (s, 9H), 1.96 (m, 1H), 1.98 (s, 1H), 3.34 (dd, J=4.0, 14.6 Hz, 1H), 3.42 (d, J=12.0 Hz, 1H), 3.55 (m, 1H), 3.71 (m, 1H), 4.11 (m, 1H), 4.27 (m, 1H), 4.91 (dd, J=0.8 Hz, 12.0 Hz, 1H).

### EXAMPLE 2

### (2S,4R)-tert-butyl 4-(tert-butyl-dimethylsilyloxy)-2-((methylsulfonyloxy)-methyl)pyrrolidine-1-carboxylate (9)

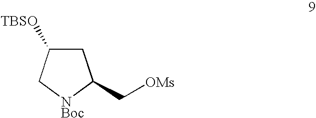

To a solution of Compound 8 (6.50 g, 19.6 mmol) and Et₃N (5.5 mL, 39.2 mmol) in CH₂Cl₂ (80 ml) was added MsCl (2.3 mL, 29.4 mmol) at 4° C. [Rosen et al., J. Med. Chem. 1988, 31, 1598]. After stirring for 3 hours at the same temperature, the mixture was poured into water and extracted with ethyl acetate (AcOEt). The organic layers were combined, washed with brine, dried over Na₂SO₄, and concentrated in vacuo to afford Compound 9 (7.80 g, 97%). ¹H NMR (400 MHz, CDCl₃): δ 0.07 (s, 6H), 0.87 (s, 9H), 1.58 (s, 9H), 2.04 (m, 1H), 3.00 (s, 3H), 3.36 (d, J=1.2 Hz, 2H), 3.51 (m, 1H), 4.18 (m, 1H), 4.29 (m, 1H), 4.37 (m, 1H), 4.55 (m, 1H).

### EXAMPLE 3

### (2R,4R)-tert-butyl 4-hydroxy-2-methylpyrrolidine-1-carboxylate (10)

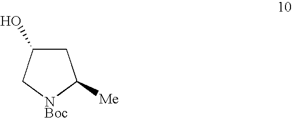

To a solution of Compound 9 (7.80 g, 24.7 mmol) in THF (20 mL) was slowly added 1 M LiBHEt₃ in THF solution (76.2 mL) at 4° C. and the mixture was allowed to warm to room temperature. After stirring for 2.5 hours, the mixture was quenched with crushed-ice and extracted with AcOEt [Rosen et al., J. Med. Chem. 1988, 31, 1598]. The organic layers were combined, washed with brine, dried over Na₂SO₄, and concentrated. The residue was dissolved in THF (100 mL) and 1 M n-Bu₄NF solution was added at 4° C. After stirring for 16 hours, the mixture was poured into water and extracted with AcOEt. The organic layers were combined, washed with brine, dried over Na₂SO₄, and concentrated in vacuo. The residue was purified by flash chromatography (hexane/AcOEt=3:1-2:1) to afford Compound 10 (3.70 g, 97%). ¹H NMR (400 MHz, CDCl₃): δ 1.23 (m, 3H), 1.47 (s, 9H), 1.55 (br, 1H) 1.74 (m, 1H), 2.10 (m, 1H), 3.44-3.49 (m, 2H), 4.00 (m, 1H), 4.40 (m, 1H). ¹³C NMR (100 MHz, CDCl₃): δ 21.20, 28.43, 42.44, 51.56, 54.28, 69.43, 79.34, 155.14. HRMS: calcd for C₁₀H₁₉NO₃ (MNa⁺) 224.1257, found 224.1255.

### EXAMPLE 4

### (2R,4R)-tert-butyl 2-methyl-4-(tosyloxy)pyrrolidine-1-carboxylate (11)

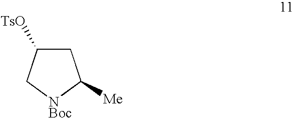

To a solution of Compound 10 (1.30 g, 6.46 mmol) in pyridine (10 mL) was added TsCl (2.22 g, 11.6 mmol) at 4° C. and the mixture was allowed to warm to room temperature [Bridges et al., J. Med. Chem. 1991, 34, 717; Heindl et al., Tetrahedron: Asymmetry 2003, 14, 3141.]. After stirring for 30 hours, the mixture was poured into a 2 N HCl solution and extracted with AcOEt. The organic layers were combined, washed with saturated NaHCO₃ solution and brine, dried over Na₂SO₄, and concentrated in vacuo. The residue was purified by flash chromatography (hexane/AcOEt=10:1-6:1) to afford Compound 11 (1.33 g, 58%). 1H NMR (400 MHz, CDCl₃): δ 1.21 (d, J=6.0, 3H), 1.44 (s, 9H), 1.74 (m, 1H), 2.26 (m, 1H), 2.46 (s, 3H), 3.41 (m, 1H), 3.62 (d, J=13.2 Hz, 1H), 3.96 (m, 1H), 4.97 (m, 1H), 7.35 (d, J=12.0 Hz, 2H), 7.78 (d, J=12.0 Hz, 2H).

### EXAMPLE 5

### (2R,4S)-tert-butyl 4-acetoxy-2-methylpyrrolidine-1-carboxylate (12)

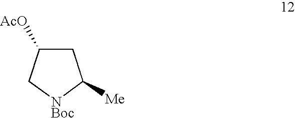

To a solution Compound 11 (1.35 g, 3.80 mmol) in toluene (15 mL) was added NH₄OAc (1.49 g, 4.94 mmol) [Bridges et al., J. Med. Chem. 1991, 34, 717; Heindl et al., Tetrahedron: Asymmetry 2003, 14, 3141]. After reflux for 4 h, the mixture was cooled to room temperature, poured into water, and extracted with AcOEt. The organic layers were combined, washed with brine, dried over Na₂SO₄, and concentrated in vacuo. The residue was purified flash column chromatography (hexane/AcOEt=10:1) to afford Compound 12 (0.91 g, 99%). ¹H NMR (400 MHz, CDCl₃): δ 1.30 (d, J=5.2 Hz, 3H), 1.47 (s, 9H), 1.77 (dd, J=0.4 Hz, 14.0 Hz, 1H), 2.07 (s, 3H), 2.30 (m, 1H), 3.46 (m, 1H), 3.65 (m, 1H), 3.97 (m, 1H), 5.23 (m, 1H).

### EXAMPLE 6

### (2R,4S)-tert-butyl 4-hydroxy-2-methylpyrrolidine-1-carboxylate (13)

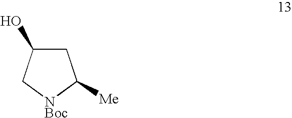

To a solution of Compound 12 (0.910 g, 3.74 mmol) in MeOH (5 mL) and THF (1 mL) was added 2 N NaOH solution (5.6 mL, 11.2 mmol) at room temperature [Heindl et al., Tetrahedron: Asymmetry 2003, 14, 3141; Zhao et al., Eur. J. Med. Chem. 2005, 40, 231]. After stirring for 30 minutes, the mixture was poured into water and extracted with AcOEt. The organic layers were combined, washed with brine, dried over Na₂SO₄, and concentrated in vacuo to afford Compound 13 (0.703 g, 93%) as a colorless solid. ¹H NMR (400 MHz, CDCl₃): δ 1.36 (d, J=6.4 Hz, 3H), 1.47 (s, 9H), 1.59 (d, J=3.2 Hz, 1H), 1.67 (d, J=13.6 Hz, 1H), 2.26 (m, 1H), 3.35 (dd, J=2.0 Hz, 12.0 Hz, 1H), 3.63 (m, 1H), 3.91(m, 1H), 4.41(m, 1H). ¹³C NMR (100 MHz, CDCl₃): δ 21.77, 28.51, 41.53, 52.49, 54.75, 77.21, 79.22, 154.52. HRMS: calcd for C₁₀H₁₉NO₃ (MNa⁺) 224.1257, found 224.1262.

### EXAMPLE 7

### (2R,4R)-tert-butyl 4-cyano-2-methylpyrrolidine-1-carboxylate (14)

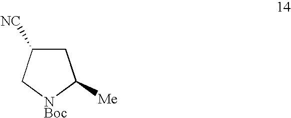

To a solution of compound 13 (0.70 g, 3.48 mmol) and Et₃N (0.97 mL, 6.96 mmol) in CH₂Cl₂ (10 mL) was added MsCl (0.40 mL, 5.22 mmol) at 4° C. [Bridges et al., J. Med. Chem. 1991, 34, 717; Heindl et al., Tetrahedron: Asymmetry 2003, 14, 3141]. After stirring for 3 hours at the same temperature, the mixture was poured into water and extracted with AcOEt. The organic layers were combined, washed with brine, dried over Na₂SO₄, and concentrated in vacuo to give the mesylated compound (0.97 g, 100%).

Without further purification, this residue was dissolved in DMSO (10 mL) and NaCN (0.256 g, 5.22 mmol) was added [Bridges et al., J. Med. Chem. 1991, 34, 717; Heindl et al., Tetrahedron: Asymmetry 2003, 14, 3141]. This mixture was stirred at 80° C. for 20 hours. The mixture was treated with saturated NaHCO₃ and extracted with AcOEt. The organic layers were combined, washed with brine, dried over Na₂SO₄, and concentrated in vacuo. The residue was purified by flash column chromatography (hexane/AcOEt=6:1) to give Compound 14 (0.422 g, 58%). ¹H NMR (400 MHz, CDCl₃): δ 1.20 (d, J=8.4 Hz, 3H), 1.47 (s, 9H), 1.97 (m, 1H), 2.36 (m, 1H), 3.13 (m, 1H), 3.64-3.72 (m, 2H), 4.06 (br, 1H). ¹³C NMR (100 MHz, CDCl₃): δ 20.18, 26.11, 28.32, 36.73, 48.98, 52.00, 80.02, 119.88, 153.59. HRMS: calcd for C₁₁H₁₈N₂O₂ (MNa⁺) 233.1260, found 233.1257.

### EXAMPLE 8

### (3R,5R)-5-methyl-3-pyrrolidine-carboxylic acid (1)

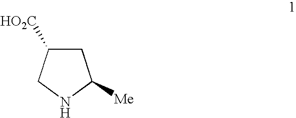

A solution of Compound 7 (0.42 g, 2.00 mmol) in conc. HCl (4.2 mL) was refluxed for 2 hours. The mixture was concentrated in vacuo. The resulting colorless solid was dissolved in water and the solution was loaded to Dowex 50WX8-100 ion-exchange resin (H⁺ form, activated with 0.01 M HCl). The resin was washed with water then eluted with 1 M ammonium hydroxide. The eluted fractions were lyophilized to afford Compound 1 (0.232 g, 90%) as a colorless solid. ¹H NMR (400 MHz, CD₃OD): δ 1.41 (d, J=8.4 Hz, 3H), 1.90 (m, 1H), 2.43 (m, 1H), 3.11 (m, 1H), 3.44 (dd, J=8.0 Hz, 11.6 Hz, 1H), 3.56 (dd, J=5.6 Hz, 11.6 Hz, 1H), 3.78 (m, 1H). ¹³C NMR (100 MHz, CD₃OD): δ 17.5, 37.6, 45.9, 49.3, 56.8, 179.7. HRMS: calcd for C₆H₁₁NO₂ (MH⁺) 130.0863, found 130.0868. [α]²⁵_{D} +10.3 (c 0.58, MeOH).

### EXAMPLE 9

### Another Route from Compound 10 to Compound 13

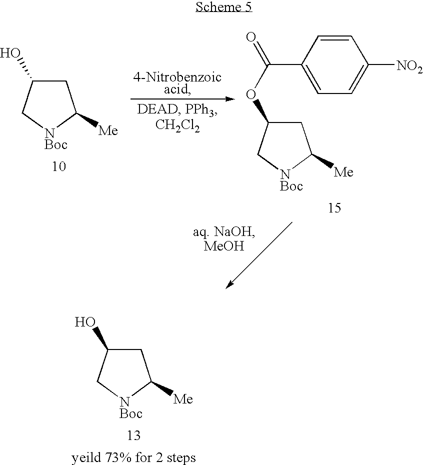

To a solution of Compound 10 (0.70 g, 3.48 mmol) and PPh₃ (1.37 g, 5.22 mmol) in CH₂Cl₂ (7 mL) was added DEAD (0.91 mL, 5.22 mmol) at 4° C. [Zhao et al., Eur. J. Med. Chem. 2005, 40, 231]. The resulting mixture was stirred for 10 min and then 4-nitrobenzoic acid (1.62 g, 5.22 mmol) was added. This mixture was allowed to warm up to room temperature and stirred for 16 hours. The reaction mixture was quenched with 2 N NaOH solution and extracted with AcOEt. The organic layers were combined, washed with brine, dried over Na₂SO₄, and concentrated. The residue was purified by flash column chromatography to give Compound 15 (0.885 g, 73%) as a pale yellow solid. Compound 15: ¹H NMR (400 MHz, CDCl₃): δ 1.38 (d, J=0.4 Hz, 3H), 1.48 (s, 9H), 1.96 (d, J=14.4 Hz, 1H), 2.47 (m, 1H), 3.64-3.83 (m, 2H), 4.11 (m, 1H), 5.55 (m, 1H), 8.21 (d, J=8.0 Hz, 2H), 8.31 (d, J=8.0 Hz, 2H).

Compound 15 (0.885 g, 2.51 mmol) was dissolved in MeOH (5 mL) and THF (5 mL) and 2 N NaOH solution was added at room temperature. After stirring for 30 minutes, the mixture was poured into water and extracted with AcOEt. The organic layers were combined, washed with brine, dried over Na₂SO₄, and concentrated in vacuo to give Compound 13 (0.52 g, 100%) as a colorless solid.

General Procedure for the Mannich-Type Reaction Between N-PMP Protected α-Imino Ethyl Glyoxylate and Aldehyde Donors (Table 2)

N-(p-Methoxy)phenyl-protected [N-PMP-protected] α-imino ethyl glyoxylate (0.25 mmol, 1 equiv) was dissolved in anhydrous DMSO (2.5 mL) and aldehyde (0.5 mmol, 2 equiv) was added, followed by catalyst Compound 1 (0.0125 mmol, 0.05 equivalents). After stirring for 0.5-3 hours at room temperature, the mixture was worked up by addition of aqueous saturated ammonium chloride solution and extracted with AcOEt (three or four times). The combined organic layers were washed with brine, dried with MgSO₄, filtered, concentrated in vacuo, and purified by flash column chromatography (10-15% AcOEt/hexane) to afford the corresponding Mannich addition product.

When the catalyst loading was 1 or 2 mol %, the reaction was performed using N-PMP-protected α-imino ethyl glyoxylate (0.5 mmol, 1 equivalents), aldehyde (1.0 mmol, 2 equiv), and catalyst Compound 1 (0.005 or 0.01 mmol, 0.01 or 0.02 equivalents) in DMSO (5 mL). The reactions were performed in a closed system (a vial with a cap). An inert atmosphere of nitrogen or argon was not necessary for the reactions.

Product Data

### Ethyl (2S,3R)-3-formyl-2-(p-methoxyphenylamino)-butanoate (2)

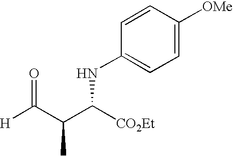

¹H NMR (400 MHz, CDCl₃): δ 1.17 (d, J=7.2 Hz, 3H, CHCH₃), 1.23 (t, J=7.2 Hz, 3H, OCH₂CH₃), 2.85-2.92 (m, 1H, CHCHO), 3.74 (s, 3H, OCH₃), 4.09 (brd, J=8.4 Hz, 1H, NHPMP), 4.14-4.23 (m, 2H, OCH₂CH₃), 4.34-4.37 (brdd, J=6.0 Hz, 8.8 Hz, 1H, CHNHPMP), 6.66 (d, J=9.0 Hz, 2H, ArH), 6.78 (d, J=9.0 Hz, 2H, ArH), 9.73 (d, J=1.2 Hz, 1H, CHCHO). ¹³C NMR (100 MHz, CDCl₃): δ 201.9, 171.8, 153.2, 140.1, 115.6, 114.9, 61.6, 58.6, 55.7, 48.5, 14.2, 9.9. HRMS: calcd for C₁₄H₂₀NO₄ (MH⁺) 266.1387, found 266.1382.

### Ethyl (E)-3-benzyloxyiminomethyl-2-(p-methoxyphenylamino)-butanoate (7)

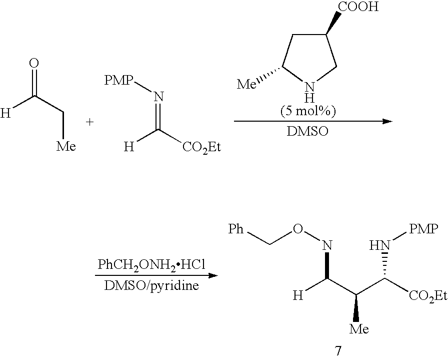

A mixture of N-PMP-protected α-imino ethyl glyoxylate (0.5 mmol, 1 equiv), an aldehyde donor (1.0 mmol, 2 equivalents), and catalyst 1 (0.025 mmol, 0.05 equivalents) in DMSO (5 mL) was stirred for 1 hour at room temperature. To the mixture, O-benzylhydroxylamine hydrochloride (1.3 mmol) and pyridine (0.5 mL) were added. The mixture was stirred for an additional 4 hours at room temperature, filtered through Celite®, and concentrated in vacuo. The residue was purified by flash column chromatography to afford oxime 7. ¹H NMR (400 MHz, CDCl₃): δ 1.18 (d, J=6.6 Hz, 3H, CHCH₃), 1.21 (t, J=7.2 Hz, 3H, OCH₂CH₃), 2.86-2.95 (m, 1H, CH₃CHCH═N), 3.74 (s, 3H, OCH₃), 3.91-3.98 (m, 2H, NHCHCO₂Et), 4.14 (q, J=7.2 Hz, 2H, OCH₂CH₃), 5.07 (s, 2H, CH₂Ph), 6.55 (d, J=9.0 Hz, 2H, ArH), 6.75 (d, J=9.0 Hz, 2H, ArH), 7.31-7.44 (m, 6H, ArH and CH═N). ¹³C NMR (100 MHz, CDCl₃): δ 172.5, 152.8, 151.8, 140.8, 137.6, 128.4, 128.2, 127.8, 115.2, 114.8, 75.7, 61.3, 61.2, 55.7, 37.5, 14.7, 14.2. HRMS: calcd for C₂₁H₂₁N₂O₄ (MH⁺) 371.1965, found 371.1966. HPLC (Daicel Chairalcel AD, hexane/i-PrOH=99:1, flow rate 1.0 mL/min, λ=254 nm): t_{R} (anti major enantiomer)=66.6 minutes, t_{R} (anti minor enantiomer)=57.8 minutes.

### Ethyl (2S,3R)-3-formyl-2-(p-methoxyphenylamino)-4-methyl-pentanoate (3)

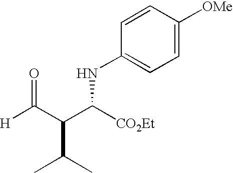

¹H NMR (300 MHz, CDCl₃): δ 1.07 (d, J=6.9 Hz, 3H, CHCH₃), 1.12 (d, J=6.9 Hz, 3H, CHCH₃), 1.21 (t, J=7.2 Hz, 3H, OCH₂CH₃), 2.02-2.18 (m, 1H, CH(CH₃)₂), 2.57-2.63 (m, 1H, CHCHO), 3.74 (s, 3H, OCH₃), 4.00 (brs, 1H, NHPMP), 4.15 (q, J=6.9 Hz, 2H, OCH₂CH₃), 4.35 (d, J=7.8 Hz, 1H, CHNHPMP), 6.66 (d, J=9.0 Hz, 2H, ArH), 6.77 (d, J=9.0 Hz, 2H, ArH), 9.75 (d, 1H, J=3.3 Hz, CHCHO). ¹³C NMR (100 MHz, CDCl₃): δ 203.2, 172.8, 153.2, 140.4, 115.9, 114.8, 61.3, 59.6, 57.2, 55.6, 27.5, 21.2, 19.2, 14.1. HRMS: calcd for C₁₆H₂₄NO₄ (MH⁺) 294.1700, found 294.1701. HPLC (Daicel Chairalcel AS-H, hexane/i-PrOH=99:1, flow rate 1.0 mL/min, λ=254 nm): t_{R} (anti major enantiomer, (2S,3R)-3)=24.0 minutes, t_{R} (anti minor enantiomer, (2R,3S)-3)=49.3 minutes. [α]²⁵_{D} −35.4 (c 1.8, CHCl₃).

### Ethyl (2S,3R)-3-formyl-2-(p-methoxyphenylamino)-octanoate (4)

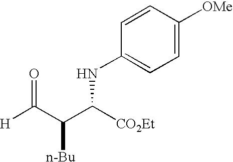

¹H NMR (300 MHz, CDCl₃): δ 0.89 (m, 3H, CH₂CH₂CH₃), 1.23 (t, J=7.2 Hz, 3H, OCH₂CH₃), 1.25-1.80 (m, 6H), 2.75 (m, 1H, CHCHO), 3.74 (s, 3H, OCH₃), 4.03 (brs, 1H, NHPMP), 4.18 (dq, J=0.9 Hz, 7.2 Hz, 2H, OCH₂CH₃), 4.26 (brd, J=6.3 Hz, 1H, CHNHPMP), 6.65 (d, J=9.0 Hz, 2H, ArH), 6.78 (d, J=9.0 Hz, 2H, ArH), 9.65 (d, J=2.4 Hz, 1H, CHCHO). ¹³C NMR (75 MHz, CDCl₃): δ 202.3, 172.2, 153.2, 140.3, 115.7, 114.8, 61.5, 58.1, 55.7, 53.9, 29.4, 25.4, 22.6, 14.2, 13.8. HRMS: calcd for C₁₇H₂₆NO₄ (MH⁺) 308.1856, found: 308.1852. HPLC (Daicel Chairalcel AS-H, hexane/i-PrOH=99:1, flow rate 1.0 mL/min, λ=254 nm): t_{R} (anti major enantiomer, (2S,3R)-4)=24.4 minutes, t_{R} (anti minor enantiomer, (2R,3S)-4)=28.5 minutes. [α]²⁵_{D} −11.0 (c 1.4, CHCl₃).

### Ethyl (2S,3R)-3-formyl-2-(p-methoxyphenylamino)-heptanoate (5)

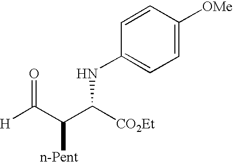

¹H NMR (400 MHz, CDCl₃): δ 0.87 (t, J=6.8 Hz, 3H, CH₂CH₃), 1.23 (t, J=7.2 Hz, 3H, OCH₂CH₃), 1.24-1.78 (m, 8H), 2.72-2.78 (m, 1H, CHCHO), 3.74 (s, 3H, OCH₃), 4.03 (brd, J=6.4 Hz, 1H, NHPMP), 4.18 (dq, J=1.6 Hz, 7.2 Hz, 2H, OCH₂CH₃), 4.26 (m, 1H, CHNHPMP), 6.65 (d, J=9.2 Hz, 2H, ArH), 6.78 (d, J=9.2 Hz, 2H, ArH), 9.65 (d, J=2.4 Hz, 1H, CHCHO). ¹³C NMR (100 MHz, CDCl₃): δ 202.3, 172.2, 153.1, 140.3, 115.7, 114.8, 61.5, 58.1, 55.6, 53.9, 31.6, 27.0, 25.6, 22.3, 14.1, 13.9. HRMS: calcd for C₁₈H₂₇NO₄ (MH⁺) 322.2013, found: 322.2007. HPLC (Daicel Chiralpak AS, hexane/i-PrOH=99:1, flow rate 1.0 mL/min, λ=254 nm): t_{R} (anti major enantiomer, (2S,3R)-5)=21.5 minutes, t_{R} (anti minor enantiomer, (2R,3S)-5)=24.9 minutes. [α]²⁵_{D} −11.9 (c 1.3, CHCl₃).

### Ethyl (2S,3R)-3-formyl-2-(p-methoxyphenylamino)-hex-5-enoate (6)

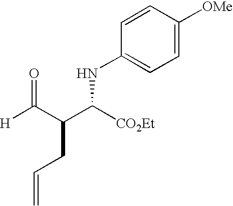

¹H NMR (400 MHz, CDCl₃): δ 1.23 (t, J=7.2 Hz, 3H, OCH₂CH₃), 2.37-2.59 (m, 2H, CH₂CH═CH₂), 2.94-2.99 (m, 1H, CHCHO), 3.74 (s, 3H, OCH₃), 4.08 (brd, J=10.0 Hz, 1H, NHPMP), 4.18 (dq, J=0.8 Hz, 7.2 Hz, 2H, OCH₂CH₃), 4.28 (m, 1H, CHNHPMP), 5.12-5.17 (m, 2H, CH═CH₂), 5.77-5.88 (m, 1H, CH═CH₂), 6.65 (d, J=9.2 Hz, 2H, ArH), 6.77 (d, J=9.2 Hz, 2H, Ar—H), 9.69 (d, J=1.6 Hz, 1H, CHCHO). ¹³C NMR (100 MHz, CDCl₃): δ 201.9, 172.2, 153.1, 140.5, 134.3, 118.2, 115.8, 114.8, 61.6, 57.7, 55.6, 53.1, 30.0, 14.1. HRMS: calcd for C₁₆H₂₂NO₄ (MH⁺) 292.1543, found: 292.1537. HPLC (Daicel Chairalcel AS-H, hexane/i-PrOH=99:1, flow rate 1.0 mL/min, λ=254 nm): t_{R} (anti major enantiomer, (2S,3R)-6)=30.2 minutes, t_{R} (anti minor enantiomer, (2R,3S)-6)=38.5 minutes. [α]²⁵_{D} +21.5 (c 1.0, CHCl₃).

Synthesis of Catalysts

### (R)-3-Pyrrolidinecarboxylic acid [(R)-16]^{1,2}

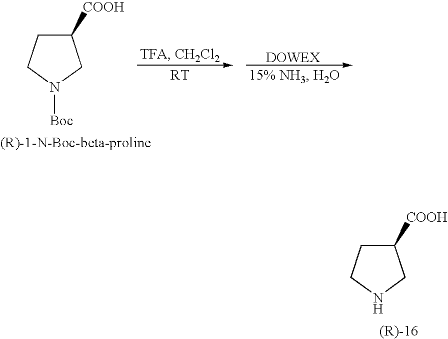

(R)-3-Pyrrolidinecarboxylic acid (also known as, (R)-pyrrolidine-3-carboxylic acid, (R)-□-proline) (CAS No. 72580-53-1) was prepared from (R)1-N-Boc-beta-proline purchased from J & W Pharmlab.

To a solution of (R)-1-N-Boc-beta-proline (2.00 g, 9.3 mmol) in CH₂Cl₂ (10 mL) was added trifluoroacetic acid (TFA) (5 mL) at 0° C., and the resulting mixture was stirred at room temperature over night (about 18 hours). The mixture was concentrated in vacuo, dissolved in water, and loaded to Dowex 50WX8-100 ion-exchange resin (H⁺ form, activated with 0.1 M HCl). The resin was washed with water then eluted with 15% ammonium hydroxide and the eluted fractions were lyophilized. The resulting semi-solid was dissolved in MeOH-toluene and the solvents were removed in vacuo to give (R)-16 (1.07 g) as a colorless solid. [(1) Mazzini et al., J. Org. Chem. 1997, 62, 5215; (2) Thomas et al., Synthesis. 1998, 10, 1491]

### (S)-3-Pyrrolidinecarboxylic acid [(S)-16]^{1,2}

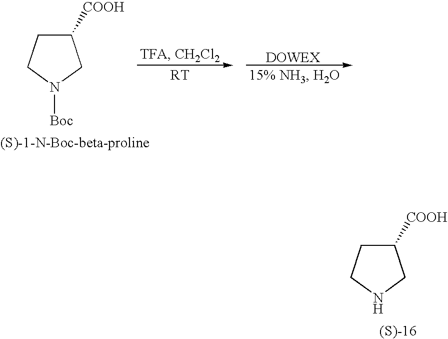

(S)-3-Pyrrolidinecarboxylic acid (also known as, (S)-pyrrolidine-3-carboxylic acid, (S)-β-proline) (CAS No. 72580-54-2) was prepared from (S)1-N-Boc-beta-proline purchased from J & W Pharmlab. To a solution of (S)-1-N-Boc-beta-proline (450 mg, 2.0 mmol) in CH₂Cl₂ (2 mL) was added TFA (2 mL) at 0° C., and the resulting mixture was stirred at room temperature for 3 hours. The mixture was concentrated in vacuo, dissolved in water, and loaded to Dowex 50WX8-100 ion-exchange resin (H⁺ form, activated with 0.1 M HCl). The resin was washed with water then eluted with 15% ammonium hydroxide and the eluted fractions were lyophilized. The resulting semi-solid was dissolved in MeOH-toluene and the solvents were removed in vacuo to give (S)-16 (232 mg) as a colorless solid. [(1) Mazzini et al., J. Org. Chem. 1997, 62, 5215; (2) Thomas et al., Synthesis. 1998, 10, 1491]

### (S)-Trifluoro-N-(pyrrolidin-3-ylmethyl)-methanesulfonamide [(S)-17]

Catalyst (S)-17 was prepared from (R)-3-aminomethyl-1-N-Boc-pyrrolidine purchased from Asta Tech, Inc.

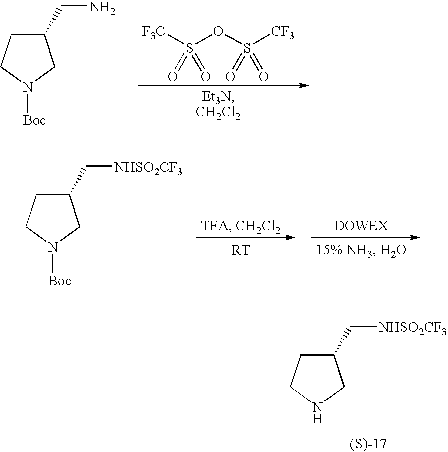

### A. (S)-1-N-Boc-3-[(trifluoromethyl-sulfonamido)methyl]pyrrolidine

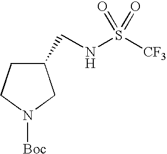

To a solution of (R)-3-aminomethyl-1-N-Boc-pyrrolidine (500 mg, 2.5 mmol) and triethylamine (1.05 mL, 7.5 mmol) in anhydrous CH₂Cl₂ (40 mL) was slowly added trifluoromethanesulfonic anhydride (0.5 mL, 3 mmol) in anhydrous CH₂Cl₂ (6 mL) using a syringe pump over 1 hour at 0° C. under N₂. [Wang et al., Tetrahedron Lett. 2004, 45, 7235] The resulting mixture was stirred over night (about 18 hours) at room temperature. The mixture was concentrated in vacuo and directly purified by flash column chromatography (EtOAc/hexane=1/2-2/1) to afford (S)-1-N-Boc-3-[(trifluoromethylsulfonamido)methyl]-pyrrolidine (556 mg, 67%) as a colorless solid. ¹H NMR (400 MHz, CDCl₃): δ 1.42 (s, 9H), 1.65 (m, 1H), 2.01 (m, 1H), 2.46 (m, 1H), 3.04-3.53 (m, 6H), 7.05 (brs, ½H, NH), 7.30 (brs, ½H, NH). ¹³C NMR (100 MHz, CDCl₃): δ 28.3, 28.7, 38.7, 39.3, 44.7, 45.2, 46.2, 46.3, 48.8, 49.0, 60.6, 65.2, 79.8, 79.9, 119.7 (q, J=310 Hz), 154.7, 154.8.

### B. (S)-Trifluoro-N-(pyrrolidin-3-ylmethyl)-methanesulfonamide [(S)-17]

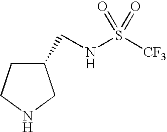

To a solution of (S)-1-N-Boc-3-[(trifluoro-methylsulfonamido)methyl]pyrrolidine (332 mg, 1.00 mmol) in CH₂Cl₂ (2 mL) was added triethylamine (1 mL) at 0° C., and the resulting mixture was stirred at room temperature for 3 hours. The mixture was concentrated in vacuo, dissolved in water, and loaded to Dowex 50WX8-100 ion-exchange resin (H⁺ form, activated with 0.1 M HCl). The resin was washed with water then eluted with 15% ammonium hydroxide. The eluted fractions were lyophilized to afford Compound (S)-17 (197 mg, 85%) as a colorless solid. ¹H NMR (500 MHz, CD₃OD): δ 1.78 (m, 1H), 2.09 (m, 1H), 2.47 (m, 1H), 3.01-3.08 (m, 2H), 3.13-3.20 (m, 2H), 3.27-3.31 (m, 2H). ¹³C NMR (125 MHz, CD₃OD): δ 29.0, 41.3, 46.7, 48.9, 50.1, 123.5 (q, J=327 Hz), HRMS: calcd for C₆H₁₂F₃N₂O₂S (MH⁺) 233.0566, found 233.0575. [α]_{D}²⁵ −7.2 (c 0.47, C₂H₅OH).
Preparation of N-PMP-Protected α-Imino Esters

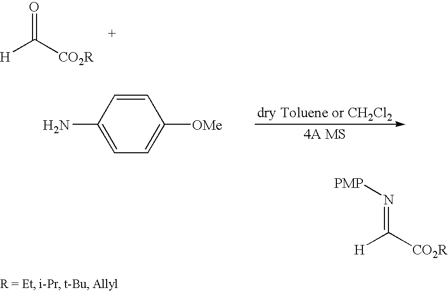

### Ethyl (E)-2-(p-methoxyphenylimino)acetate

A mixture of glyoxylic acid ethyl ester (polymer form 45-50% in toluene, 10 mL, about 45 mmol), p-anisidine (5.54 g, 45 mmol), and molecular sieves 4 Å (50 g) in anhydrous toluene (250 mL) was stirred at room temperature for 2-6 hours. After filtration through celite, the mixture was concentrated in vacuo to afford the imine. The imine was used for the Mannich-type reactions without further purification. [Juhl et al., Angew. Chem., Int. Ed. 2001, 40, 2995]

The following glyoxylic acid esters were prepared by the reported procedures. Generally, mixture of a glyoxylic acid ester (12 mmol) and p-anisidine (11.5 mmol) in CH₂Cl₂ (30 mL) was stirred at room temperature for 30 minutes. The mixture was concentrated in vacuo to afford the imine. The imine was used without further purification.
Allyl (E)-(p-methoxyphenylimino)acetate [Guthikonda et al., J. Med. Chem. 1987, 30, 871; Katherine, E. U.S. Pat. No. 4,695,626; Cozzi et al., Chirality 1998, 10, 91]
tert-buty (E)-(p-methoxyphenylimino)acetate [Våbenø et al., Org. Chem. 2002, 67, 9186; Palomo et al., J. Org. Chem. 1999, 64, 1693]
isopropyl (E)-(p-methoxyphenylimino)acetate [Juhl et al., Angew. Chem., Int. Ed. 2001, 40, 2995]
   Direct Asymmetric Anti-Mannich-Type Reactions of Unmodified Ketones

A. General Procedure for the (R)-15-Catalyzed Mannich-Type Reactions Between Unmodified Ketones and α-Imino Esters (Table 4)

The reactions were performed in a closed system (a vial with a cap). An inert atmosphere of nitrogen or argon was not necessary. N-PMP-protected α-imino ester (0.5 mmol, 1.0 equiv.) was dissolved in 2-PrOH (1.0 mL) and ketone (5.0 mmol, 10 equiv.) was added to the solution, followed by catalyst Compound (R)-15 (0.05 mmol, 0.1 equiv.). After stirring at room temperature (25° C.) for the indicated time in the Table, the reaction mixture was concentrated in vacuo and purified by flash column chromatography. The anti- and syn-isomers of the Mannich product shown in Table 4 were not discriminated on TLC each other (see below for preparation of (±)-anti- and syn-products). The diastereomeric ratio was determined by ¹H NMR of the isolated product. The enantiomeric excess of the anti-product was determined by chiral-phase HPLC analysis. The chiral-phase HPLC analysis was also used for the determination of the diastereomeric ratio as indicated.

B. General Procedure for Compound (R)-16-Catalyzed Mannich-Type Reactions Between Unmodified Cyclic Ketones and α-Imino Esters (Table 5)

The reactions were performed in a closed system (a vial with a cap). An inert atmosphere of nitrogen or argon was not necessary. N-PMP-protected α-imino ester (0.5 mmol, 1.0 equiv.) was dissolved in 2-PrOH (1.0 mL) and ketone (1.0 mmol, 2.0 equiv.) was added to the solution, followed by catalyst Compound (R)-16 (0.05 mmol, 0.1 equiv or 0.025 mmol, 0.05 equiv.). After stirring at room temperature (25° C.) for 10-16 hours, the reaction mixture was concentrated in vacuo and purified by flash column chromatography. The anti- and syn-isomers of the Mannich product shown in Table 5 were not discriminated on TLC each other, except for Compounds 23 and 29. The diastereomeric ratio was determined by ¹H NMR of the isolated product. The enantiomeric excess of the anti-product was determined by chiral-phase HPLC analysis.

C. Synthesis of (±)-Anti- and (±)-Syn-Mannich Products

Racemic standards of the anti-Mannich products were synthesized by using (±)-3-pyrrolidinecarboxylic acid (CAS No. 59378-87-9) purchased from J & W Pharmlab as catalysts. Racemic standards of the syn-Mannich products were synthesized by using (±)-proline as catalyst. Alternatively, a racemic mixture of the diastereomers and enantiomers was synthesized using pyrrolidine-trifluoroacetic acid as catalyst. These reactions are shown below.

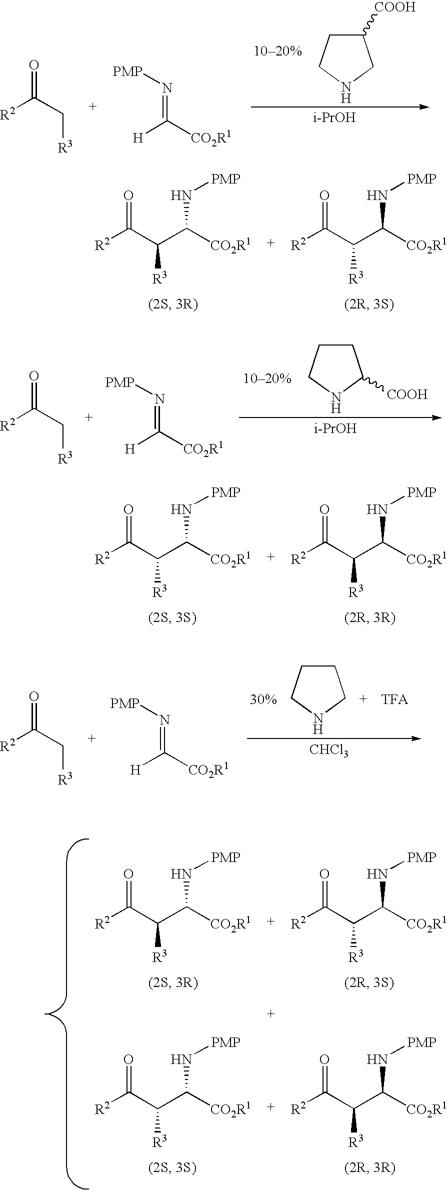

### Ethyl (2S,3R)-2-(p-methoxyphenylamino)-3-methyl-4-oxohexanoate (17)

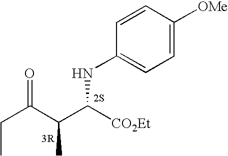

¹H NMR (400 MHz, CDCl₃): δ 1.06 (t, 3H, J=7.3 Hz, COCH₂CH₃), 1.17 (d, 3H, J=7.2 Hz, COCHCH₃), 1.21 (t, J=7.2 Hz, 3H, OCH2CH3), 2.54 (dq, 2H, J=0.8 Hz, 7.2 Hz, COCH₂CH₃), 3.02 (quintet, 1H, J=7.1 Hz, COCHCH₃), 3.73 (s, 3H, OCH₃), 4.09-4.17 (m, 3H, CHNHPMP, OCH₂CH₃), 4.19 (brd, 1H, J=7.5 Hz, CHNHPMP), 6.65 (d, 2H, J=9.0 Hz, ArH), 6.76 (d, 2H, J=9.0 Hz, ArH), ¹³C NMR (100 MHz, CDCl₃): 7.5, 13.4, 14.1, 34.9, 48.4, 55.6, 60.8, 61.2, 114.8, 115.8, 140.8, 153.0, 172.8, 212.2. HRMS: calcd for C₁₆H₂₄NO₄ (MH⁺) 294.1700, found 294.1698. HPLC (Daicel Chiralpak AS, hexane/i-PrOH=99:1, 1.0 mL/min, λ=254 nm): t_{R} (anti major enantiomer)=20.0 min, t_{R} (anti minor enantiomer)=37.7 min. [α]_{D}²⁵ −27.8 (c 2.9, CHCl₃, 98% ee).

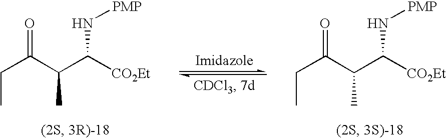

Imidazole isomerization [Ward et al., J. Org. Chem. 2004, 69, 4808] of the anti-18 obtained from the (R)-16-catalyzed reaction afforded the syn-product possessing a (2S,3S) configuration, which was the product of the (S)-proline-catalyzed reaction. [Cordova et al., J. Am. Chem. Soc. 2002, 124, 1842] This result confirmed that the major anti-product Compound 18 generated from the (R)-16-catalyzed reaction had a (2S,3R) configuration.

### tert-Butyl (2S,3R)-2-(p-methoxyphenylamino)-3-methyl-4-oxohexanoate (19)

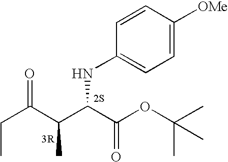

¹H NMR (500 MHz, CDCl₃): δ 1.07 (t, 3H, J=7.2 Hz, COCH₂CH₃), 1.15 (d, 3H, J=7.1 Hz, COCHCH₃), 1.38 (s, 9H, CH(CH₃)₃), 2.54 (dq, 1H, J=0.8 Hz, 7.2 Hz, COCHHCH₃), 2.59 (dq, 1H, J=0.8 Hz, 7.2 Hz, COCHHCH₃), 2.97 (quintet, 1H, J=7.1 Hz, COCHCH₃), 3.73 (s, 3H, OCH₃), 4.10 (brs, 1H, CHNHPMP), 4.15 (d, 1H, J=5.9 Hz, CHNHPMP), 6.64 (d, 2H, J=9.0 Hz, ArH), 6.76 (d, 2H, J=9.0 Hz, ArH), ¹³C NMR (125 MHz, CDCl₃): δ 7.5, 12.7, 27.9, 34.7, 48.3, 55.6, 60.9, 82.1, 114.7, 115.6, 140.8, 152.9, 171.5, 211.9. HRMS: calcd for C₁₈H₂₈NO₄ (MH⁺) 322.2013, found 322.2015. HPLC (Daicel Chiralpak AS, hexane/i-PrOH=99:1, 1.0 mL/min, λ=254 nm): t_{R} (anti major enantiomer)=10.3 min; t_{R} (anti minor enantiomer)=17.9 min. [α]_{D}²⁵−45.0 (c 2.1, CHCl₃, 95%6 ee).

### Ethyl (2S,3R)-3-ethyl-2-(p-methoxyphenylamino)-4-oxoheptanoate (20)

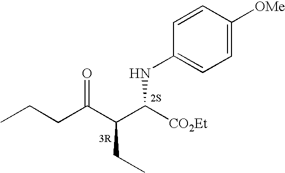

¹H NMR (500 MHz, CDCl₃): δ 0.90 (t, 3H, J=7.4 Hz, COCH₂CH₂CH₃), 0.92 (t, 3H, J=7.4 Hz, COCHCH₂CH₃), 1.18 (t, 3H, J=7.1 Hz, OCH₂CH₃), 1.54-1.76 (m, 4H, COCH₂CH₂CH₃, COCHCH₂CH₃), 2.44 (dt, 1H, J=7.1 Hz, 17.5 Hz, COCHHCH₂CH₃), 2.49 (dt, 1H, J=7.1 Hz, 17.5 Hz, COCHHCH₂CH₃), 2.87 (dt, 1H, J=6.3 Hz, 8.3 Hz, COCHCH₂CH₃), 3.72 (s, 3H, OCH₃), 4.08-4.17 (m, 4H, OCH₂CH₃, CHNHPMP, CHNHPMP), 6.62 (d, 2H, J=9.0 Hz, ArH), 6.75 (d, 2H, J=9.0 Hz, ArH), ¹³C NMR (125 MHz, CDCl₃) δ 11.9, 13.6, 14.1, 16.6, 22.0, 45.5, 55.5, 55.6, 59.6, 61.1, 114.7, 115.7, 140.9, 153.0, 173.2, 212.1. HRMS: calcd for C₁₈H₂₈NO₄ (MH⁺) 322.2013, found 322.2014. HPLC (Daicel Chiralpak AD, hexane/i-PrOH=90:10, 1.0 mL/min, λ=254 nm): t_{R} (anti major enantiomer)=7.7 min; t_{R} (anti minor enantiomer)=9.4 min.

### Ethyl (2S,3R)-2-(p-methoxyphenylamino)-3-methyl-4-oxopentanoate (21)

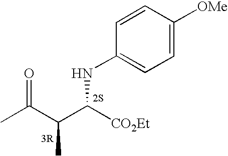

¹H NMR (400 MHz, CDCl₃): δ 1.19 (d, 3H, J=7.1 Hz, COCHCH₃), 1.21 (t, 3H, J=7.2 Hz, OCH₂CH₃), 2.22 (s, 3H, CH₃CO), 3.02 (quintet, 1H, J=7.1 Hz, COCHCH₃), 3.74 (s, 3H, OCH₃), 4.08-4.22 (m, 4H, CHNHPMP, OCH₂CH₃, CHNHPMP), 6.65 (d, 2H, J=8.9 Hz, ArH), 6.76 (d, 2H, J=8.9 Hz, ArH). ¹³C NMR (125 MHz, CDCl₃): δ 13.0, 14.1, 28.6, 49.4, 55.7, 60.5, 61.3, 114.8, 115.8, 140.7, 153.1, 172.5, 209.5. HRMS: calcd for C₁₅H₂₂NO₄ (MH⁺) 280.1543, found 280.1542. HPLC (Daicel Chiralcel AS-H, hexane/i-PrOH=96:4, 1.0 mL/min, λ=254 nm): t_{R} (anti major enantiomer)=17.1 min; t_{R} (anti minor enantiomer)=28.9 min. [α]_{D}²⁵ −20.7 (c 1.4, CHCl₃, 99% ee). The Flack parameter from X-ray crystallographic analysis is −0.4 (19), indicating that this structure shows relative stereochemistry.

### Ethyl (2S,3R)-3-ethyl-2-(p-methoxyphenylamino)-4-oxopentanoate (22)

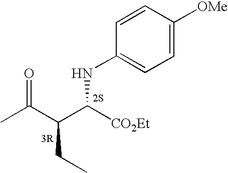

¹H NMR (500 MHz, CDCl₃): δ 0.93 (t, 3H, J=7.4 Hz, COCHCH₂CH₃)₁ 1.19 (t, 3H, J=7.1 Hz, OCH₂CH₃), 1.57-1.76 (m, 2H, CH₃CH₂CHCO), 2.20 (s, 3H, COCH₃), 2.88 (m, 1H, COCHCH₂CH₃), 3.73 (s, 3H, OCH₃), 4.10-4.17 (m, 4H, CHNHPMP, OCH₂CH₃, CHNHPMP), 6.63 (d, 2H, J=8.9 Hz, ArH), 6.75 (d, 2H, J=8.9 Hz, ArH); ¹³C NMR (125 MHz, CDCl₃): δ 11.9, 14.1, 21.8, 30.0, 55.6, 56.4, 59.5, 61.2, 114.8, 115.7, 140.8, 153.1, 173.0, 210.0. HRMS: calcd for C₁₆H₂₄NO₄ (MH⁺) 294.1700, found 294.1687. HPLC (Daicel Chiralpak AS, hexane/i-PrOH=90:10, 1.0 mL/min, λ=254 nm): t_{R} (anti major enantiomer)=8.1 min; t_{R} (anti minor enantiomer)=9.9 min.

### Ethyl (2S,3R)-3-allyl-2-(p-methoxyphenylamino)-4-oxopentanoate (23)

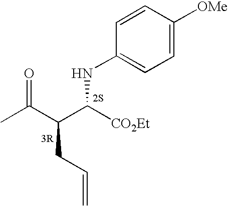

¹H NMR (400 MHz, CDCl₃): δ 1.20 (t, J=7.2 Hz, 3H, OCH₂CH₃), 2.20 (s, 3H, COCH₃), 2.35 (m, 1H, CHHCH═CH₂), 2.46 (m, 1H, CHHCH═CH₂), 3.10 (q, 1H, J=6.8 Hz, CH₃COCH), 3.73 (s, 3H, OCH₃), 4.10-4.20 (m, 4H, CHNHPMP, OCH₂CH₃, CHNHPMP), 5.06-5.11 (m, 2H, CH₂═CH), 5.75 (m, 1H, CH₂═CH), 6.63 (d, 2H, J=8.9 Hz, ArH), 6.75 (d, 2H, J=8.9 Hz, ArH); ¹³C NMR (100 MHz, CDCl₃): δ 14.1, 30.4, 32.8, 54.2, 55.6, 59.3, 61.3, 114.7, 115.8, 118.0, 134.5, 140.9, 153.0, 172.9, 209.5. HRMS: calcd for C₁₇H₂₄NO₄ (MH⁺) 306.1700, found 306.1690. HPLC (Daicel Chiralpak As, hexane/i-PrOH=90:10, 1.0 mL/min, λ=254 nm): t_{R} (anti minor enantiomer)=8.6 min, t_{R} (anti major enantiomer)=10.5 min.

### Ethyl (2S,3R)-3-acetyl-6-chloro-2-(p-methoxyphenylamino)hexanoate (24)

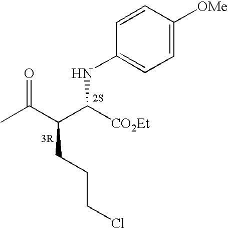

¹H NMR (400 MHz, CDCl₃): δ 1.21 (t, 3H, J=7.2 Hz, OCH₂CH₃), 1.70-1.86 (m, 4H, CH₂CH₂CH₂Cl), 2.23 (s, 3H, COCH₃), 2.97 (m, 1H, COCHCH₂), 3.51 (t, 2H, J=6.1 Hz, CH₂Cl), 3.74 (s, 3H, OCH₃), 4.09-4.18 (m, 4H, CHNHPMP, OCH₂CH₃, CHNHPMP), 6.63 (d, 2H, J=8.9 Hz, ArH), 6.76 (d, 2H, J=8.9 Hz, ArH). ¹³C NMR (125 MHz, CDCl₃): δ 14.2, 25.4, 29.7, 30.1, 44.3, 54.1, 55.7, 59.6, 61.4, 114.8, 115.8, 140.5, 153.2, 172.6, 209.2. HRMS: calcd for C₁₇H₂₅ClNO₄ (MH⁺) 342.1467, found 342.1466. HPLC (Daicel Chiralpak AD, hexane/i-PrOH=94:6, 1.0 mL/min, λ=254 nm): t_{R} (anti minor enantiomer)=19.6 min; t_{R} (anti major enantiomer)=25.5 min.

### Ethyl (2S,1′R)-2-(p-methoxyphenylamino)-2-(2′-oxocyclohexyl)acetate (24)

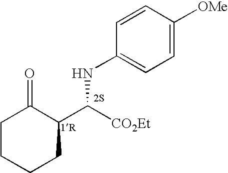

¹H NMR (400 MHz, CDCl₃): δ 1.21 (t, 3H, J=7.2 Hz, OCH₂CH₃), 1.61-1.79 (m, 2H, CH₂), 1.87-1.97 (m, 2H, CH₂), 2.02-2.14 (m, 2H, CH₂), 2.28-2.46 (m, 2H, CH₂CH₂CO), 3.08-3.13 (m, 1H, CH₂CHCO), 3.73 (s, 3H, OCH₃), 3.99 (brs, 1H, NH), 4.15 (m, 2H, OCH₂CH₃), 4.24 (m, 1H, CHNHPMP), 6.63 (d, 2H, J=8.8 Hz, ArH), 6.76 (d, 2H, J=8.8 Hz, ArH). ¹³C NMR (100 MHz, CDCl₃): δ 14.1, 24.5, 26.8, 30.5, 41.8, 53.6, 55.7, 59.1, 61.2, 114.7, 115.6, 142.1, 152.7, 173.0, 210.9. HRMS: calcd for C₁₇H₂₄NO₄ (MH⁺) 306.1700, found 306.1697. HPLC (Daicel Chiralpak AS, hexane/i-PrOH=90:10, 1.0 mL/min, λ=254 nm): t_{R} (anti major enantiomer)=12.7 min; t_{R} (anti minor enantiomer)=19.0 min. [α]_{D}²⁵ +29.1 (c 2.0, CHCl₃, 96% ee).

### Isopropyl (2S,1′R)-2-(p-methoxyphenylamino)-2-(2′-oxocyclohexyl)acetate (25)

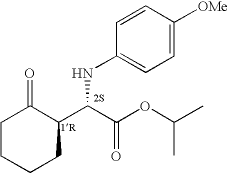

¹H NMR (500 MHz, CDCl₃): δ 1.14 (d, 3H, J=6.3 Hz, CH(CH₃)₂), 1.22 (d, 3H, J=6.3 Hz, CH(CH₃)₂), 1.63-1.78 (m, 2H, CH₂), 1.87-1.98 (m, 2H, CH₂), 2.02-2.13 m, 2H, CH₂), 2.28-2.45 (m, 2H, CH₂CH₂CO), 3.07 (m, 1H, CH₂CHCO), 3.73 (s, 3H, OCH₃), 3.96 (brd, 1H, J=3.8 Hz), 4.21 (brs, 1H), 4.99 (septet, 1H, J=6.3 Hz, CH(CH₃)₂), 6.63 (d, 2H, J=8.9 Hz, ArH), 6.75 (d, 2H, J=8.9 Hz, ArH); ¹³C NMR (125 MHz, CDCl₃): δ 21.7, 24.5, 26.9, 30.5, 41.8, 53.5, 55.7, 59.2, 68.8, 114.7, 115.7, 142.2, 152.8, 172.5, 210.8. HRMS: calcd for C₁₈H₂₆NO₄ (MH⁺) 320.1856, found 320.1854. HPLC (Daicel Chiralpak AS, hexane/i-PrOH=90:10, 1.0 mL/min, λ=254 nm): t_{R} (anti major enantiomer)=8.6 min; t_{R} (anti minor enantiomer)=14.1 min. [α]_{D}²⁵ +37 (c 1.0, CHCl₃, 94% ee).

### tert-Butyl (2S,1′R)-2-(p-methoxyphenylamino)-2-(2′-oxocyclohexyl)acetate (26)

The absolute stereochemistry of product 26 generated by the (R)-16 catalyzed reaction was determined to be (2S,1′R) by the X-ray structural analysis. The Flack parameter is 0.0 (14).

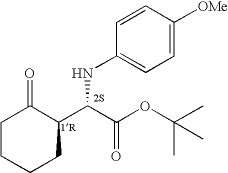

¹H NMR (500 MHz, CDCl₃): δ 1.38 (s, 9H, OC(CH₃)₃) 1.62-1.76 (m, 2H, CH₂), 1.85-1.95 (m, 2H, CH₂), 1.99-2.13 (m, 2H, CH₂), 2.27-2.44 (m, 2H, CH₂CH₂CO), 3.02 (m, 1H, CH₂CHCO), 3.73 (s, 3H, OCH₃), 3.92 (brs, 1H, NH), 4.17 (m, 1H, CHNHPMP), 6.61 (d, 2H, J=8.9 Hz, ArH), 6.74 (d, 2H, J=8.9 Hz, ArH); ¹³C NMR (100 MHz, CDCl₃): δ 24.4, 26.8, 27.9, 30.3, 41.7, 53.4, 55.6, 59.4, 81.5, 114.7, 115.4, 142.3, 152.6, 172.0, 210.7. HRMS: calcd for C₁₉H₂₈NO₄ (MH⁺) 334.2013, found 334.2012. HPLC (Daicel Chiralpak AS, hexane/i-PrOH=90:10, 1.0 mL/min, =254 nm): t_{R} (anti major enantiomer)=5.9 min; t_{R} (anti minor enantiomer)=8.4 min. [α]_{D}²⁵ +23.7 (c 3.5, CHCl₃, 95% ee).

### Allyl (2S,1′R)-2-(p-methoxyphenylamino)-2-(2′-oxocyclohexyl)acetate (27)

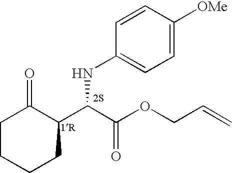

¹H NMR (500 MHz, CDCl₃) δ 1.63-1.78 (m, 2H, CH₂), 1.89-1.97 (m, 2H, CH₂), 2.02-2.14 (m, 2H, CH₂), 2.29-2.45 (m, 2H, CH₂CH₂CO), 3.13 (m, 1H, CH₂CHCO), 3.73 (s, 3H, OCH₃), 4.02 (brs, 1H, NH), 4.25 (m, 1H, CHNHPMP), 4.58 (m, 2H, OCH₂CH═CH₂), 5.17-5.21 (m, 2H, OCH₂CH═CH₂), 5.84 (m, 1H, OCH₂CH═CH₂), 6.63 (d, 2H, J=9.0 Hz, ArH), 6.76 (d, 2H, J=9.0 Hz, ArH); ¹³C NMR (100 MHz, CDCl₃): δ 24.6, 26.8, 30.5, 41.8, 53.6, 55.7, 59.0, 65.7, 114.8, 115.6, 118.2, 131.8, 142.1, 152.8, 172.7, 210.9. HRMS: calcd for C₁₈H₂₄NO₄ (MH⁺) 318.1700, found 318.1701. HPLC (Daicel Chiralpak AS, hexane/i-PrOH=90:10, 1.0 mL/min, λ=254 nm): t_{R} (anti major enantiomer)=12.6 min; t_{R} (anti minor enantiomer)=18.7 min. [α]_{D}²⁵ +25.9 (c 1.4, CHCl₃, 95% ee).

### Ethyl (2S,3′R)-2-(p-methoxyphenylamino)-2-(4′-oxotetrahydrothiopyran-3′-yl)acetate (28)

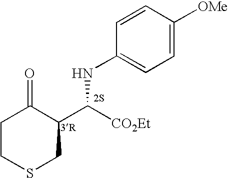

¹H NMR (500 MHz, CDCl₃) δ 1.22 (t, 3H, J=7.2 Hz, OCH₂CH₃), 2.67-2.80 (m, 2H, COCH₂CH₂S), 2.88-3.00 (m, 3H, CH₂SCHH), 3.14 (dd, J=10.0 Hz, 13.5 Hz, CH₂SCHH), 3.37 (dt, 1H, J=5.0 Hz, 10.0 Hz, COCHCH₂S), 3.73 (s, 3H, OCH₃), 4.10-4.20 (m, 3H, NH, OCH₂CH₃), 4.25 (m, 1H, CHNHPMP), 6.65 (d, 2H, J=8.9 Hz, ArH), 6.76 (d, 2H, J=8.9 Hz, ArH), ¹³C NMR (125 MHz, CDCl₃): δ 14.1, 29.8, 32.8, 43.7, 55.2, 55.7, 59.0, 61.4, 114.8, 115.9, 141.4, 153.1, 172.2, 208.0. HRMS: calcd for C₁₆H₂₂NO₄S (MH⁺) 324.1264, found 324.1263. HPLC (Daicel Chairalcel AS, hexane/i-PrOH=90:10, 1.0 mL/min, λ=254 nm): t_{R} (anti major enantiomer)=32.4 min; t_{R} (anti minor enantiomer)=46.0 min. [α]_{D}²⁵ +48.0 (c 2.6, CHCl₃, 99% ee).

### Ethyl (2S,3′S)-2-(p-methoxyphenylamino)-2-(4′-oxotetrahydropyran-3′-yl)acetate (29)

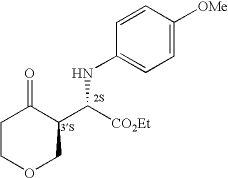

¹H NMR (400 MHz, CDCl₃) δ 1.22 (t, 3H, J=7.2 Hz, OCH₂CH₃), 2.48 (dt, 1H, J=3.9 Hz, 14.8 Hz, COCHHCH₂), 2.57-2.65 (m, 1H, COCHHCH₂), 3.24 (m, 1H, COCHCH₂O), 3.74 (s, 3H, OCH₃), 3.81 (ddd, 1H, J=3.9 Hz, 9.9 Hz, 11.4 Hz, CHHOCH₂), 3.91 (dd, 1H, J=9.0 Hz, 11.3 Hz CHHOCH₂), 4.06-4.25 (m, 6H, CH₂OCH₂, CHNHPMP, OCH₂CH₃, CHNHPMP), 6.62 (d, 2H, J=8.8 Hz, ArH), 6.77 (d, 2H, J=8.8 Hz, ArH). ¹³C NMR (100 MHz, CDCl₃): δ 14.10, 42.08, 53.8, 55.7, 56.4, 61.5, 67.8, 70.1, 114.8, 115.9, 141.3, 153.2, 172.1, 206.2. HRMS: calcd for C₁₆H₂₂NO₅ (MH⁺) 308.1492, found 308.1492. HPLC (Daicel Chairalcel AS, hexane/i-PrOH=80:20, 1.0 mL/min, λ=254 nm): t_{R} (anti major enantiomer) 16.8 min; t_{R} (anti minor enatiomer)=21.4 min.

### Ethyl (2S,1′R)-2-(p-methoxyphenylamino)-2-(5′,5′-ethylenedioxy-2′-oxocyclohexyl)acetate (30)

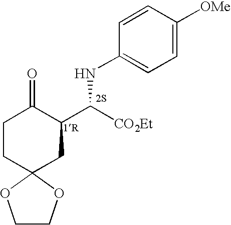

¹H NMR (400 MHz, CDCl₃) δ 1.21 (t, 3H, J=7.2 Hz, OCH2CH₃), 1.94-2.13 (m, 3H), 2.29 (t, 1H, J=13.1 Hz), 2.38-2.43 (m, 1H), 2.63-2.72 (m, 1H), 3.45-3.51 (m, 1H), 3.73 (s, 3H, OCH₃), 3.91 (brs, 1H, NH), 4.00-4.09 (m, 4H, OCH₂CH₂O), 4.14 (m, 2H, OCH₂CH₃), 4.21 (m, 1H, CHNHPMP), 6.62 (d, 2H, J=8.8 Hz, ArH), 6.75 (d, 2H, J=8.8 Hz, ArH). ¹³C NMR (100 MHz, CDCl₃): δ 14.1, 33.9, 37.6, 38.0, 50.0, 55.7, 59.0, 61.3, 64.7, 64.8, 107.5, 114.8, 115.9, 142.1, 152.9, 172.7, 209.6. HRMS: calcd for C₁₉H₂₆NO₆ (MH⁺) 364.1755, found 364.1756. HPLC (Daicel Chairalcel AS, hexane/i-PrOH=90:10, 1.0 mL/min, λ=254 nm): t_{R} (anti major enantiomer)=25.3 min; t_{R} (anti minor enatiomer)=31.0 min. [α]_{D}²⁵ +18.1 (c 1.0, CHCl₃, 97% ee).

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A compound corresponding in structure to Formula I, wherein
R is a substituent containing a hydrogen bond-forming atom within three atoms from the ring carbon to which the substituent is bonded;
X is CH₂, O, S or NR¹, wherein R¹ is a hydrocarbyl group or an amino-protecting group having one to about 18 carbon atoms;
R² is hydrido or a hydrocarbyl group containing one to about eight carbon atoms; and
R³ is hydrido or methyl, but both R² and R³ are not hydrido when X is CH₂

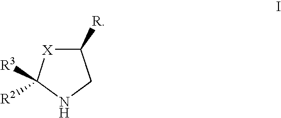

2. The compound according to claim 1 wherein R² is a C₁-C₆ alkyl group.

3. The compound according to claim 1 wherein R is a carboxyl group.

4. A compound corresponding in structure to Formula II, wherein
X is CH₂, O, S or NR¹, wherein R¹ is a hydrocarbyl group or a hydrocarbyloxy group having one to about 18 carbon atoms;
R² is hydrido or a hydrocarbyl group containing one to about eight carbon atoms; and
R³ is hydrido or methyl, but both R² and R³ are not hydrido when X is CH₂

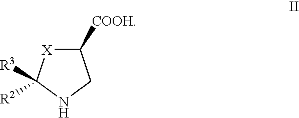

5. The compound according to claim 4 wherein R² is a C₁-C₆ alkyl group.

6. The compound according to claim 5 wherein said C₁-C₆ alkyl group is a methyl group.

7. The compound according to claim 4 wherein R³ is methyl.

8. The compound according to claim 4 wherein R³ is hydrido.

9. The compound according to claim 4 wherein X is S.

10. The compound according to claim 4 wherein X is NR¹.

11. The compound according to claim 10 wherein R¹ is a hydrocarbyl group.

12. The compound according to claim 4 wherein X is CH₂.

13. A compound corresponding in structure to Formula III, wherein
X is CH₂, O, S or NR¹, wherein R¹ is a hydrocarbyl group or a hydrocarbyloxy group having one to about 18 carbon atoms; and
R² is a hydrocarbyl group containing one to about eight carbon atoms

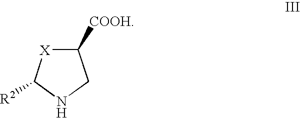

14. The compound according to claim 13 wherein R² is a C₁-C₆ alkyl group.

15. The compound according to claim 14 wherein said C₁-C₆ alkyl group is a methyl group.

16. The compound according to claim 14 wherein X is S.

17. The compound according to claim 14 wherein X is NR¹.

18. The compound according to claim 17 wherein R¹ is a hydrocarbyl group.

19. The compound according to claim 14 wherein X is CH₂.

20. A method for asymmetrically forming a β-aminoaldehyde or β-aminoketone diastereomeric products having two chiral centers on adjacent carbon atoms and in which the anti-diastereomers are in excess over the syn-diastereomers comprising the steps of:
(a) admixing an excess of an enolizable donor aldehyde or ketone molecule with an acceptor molecule having an imino group (>C═N—) whose carbon atom is bonded directly to a second carbon (the alpha carbon) that has one or no bonded hydrogen atoms, wherein the donor and acceptor molecules are dissolved or dispersed in a liquid solvent and are in the presence of a chiral amine catalyst to form an addition product reaction medium, and wherein said chiral amine catalyst corresponds in structure to a compound of Formula X, wherein
R is a substituent containing a hydrogen bond-forming atom within three atoms from the ring carbon to which the substituent is bonded;
X is CH₂, O, S or NR¹, wherein R¹ is a hydrocarbyl group or an amino-protecting group having one to about 18 carbon atoms;
R² is hydrido or a hydrocarbyl group containing one to about eight carbon atoms; and
R³ is hydrido or methyl 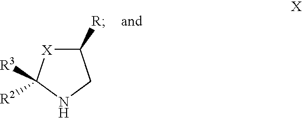
(b) maintaining the reaction medium for a time sufficient to form a β-aminoaldehyde or β-aminoketone diastereomeric products having two chiral centers on adjacent carbon atoms and in which the anti-diastereomers are in excess over the syn-diastereomers.

21. The method according to claim 20 wherein said donor molecule contains 2 to about 28 carbon atoms exclusive of any carbon atoms that may be present in the diprotectedamino group.

22. The method according to claim 20 wherein said acceptor molecule contains 2 to about 30 carbon atoms exclusive of carbon atoms present bonded to the nitrogen of the imino group.

23. The method according to claim 20 wherein said chiral amine catalyst contains up to about 20 carbon atoms.

24. The method according to claim 20 wherein said chiral amine catalyst is present in an amount of about 0.1 to about 50 mole percent of the amount of the acceptor aldehyde or ketone.

25. The method according to claim 20 wherein said solvent that is a liquid at a temperature of about −50° C. to about 150° C.

26. The method according to claim 20 including the further step of recovering the β-aminoaldehyde or β-aminoketone products.

27. The method according to claim 20 wherein said chiral amine catalyst contains up to about 20 carbon atoms.

28. The method according to claim 20 wherein R is a carboxyl group.

29. The method according to claim 20 wherein R² is a C₁-C₆ alkyl group.

30. The method according to claim 20 wherein said C₁-C₆ alkyl group is a methyl group.

31. The method according to claim 20 wherein X is S.

32. The method according to claim 20 wherein X is NR¹.

33. The method according to claim 32 wherein R¹ is a hydrocarbyl group.

34. The method according to claim 32 wherein X is CH₂.

35. The method according to claim 20 wherein the donor molecule has a structure that corresponds to the formula 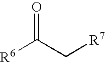
wherein R⁷ is selected from the group consisting of hydrido, C₁-C₈ straight chain, branched chain or cyclic hydrocarbyl, halogen, cyano, hydroxy, C₁-C₈-acyloxy, C₁-C₈-hydrocarbyloxy, C₁-C₈-hydrocarbylthio, azido, phthalimido and trifluoromethyl groups;
R⁶ is selected from the group consisting of hydrido, a C₁-C₁₈ straight chain, branched chain or cyclic hydrocarbyl group, an aryl group, and an aryl group substituted with a substituent selected from the group consisting of C₁-C₈ straight chain, branched chain or cyclic hydrocarbyl group, halogen, cyano, trifluoromethyl, nitro, hydroxyl, and a —CO₂R^{a} group, wherein R^{a} is a C₁-C₈ straight chain, branched chain or cyclic hydrocarbyl group; or
R⁶ and R⁷ together with the depicted —C(O)—CH₂— group form a cyclic structure that contains 5 to about 9 atoms in the ring, including up to two heteroatoms that are one or both of oxygen and sulfur.

36. The method according to claim 35 wherein the cyclic donor molecule structure has an even number of ring atoms.

37. The method according to claim 36 wherein the cyclic donor molecule has only one heteroatom present.

38. The method according to claim 37 wherein the one heteroatom of the donor molecule is located symmetrically two or three carbon atoms away from the depicted carbonyl group.

39. The method according to claim 35 wherein the cyclic donor molecule structure has an odd number of atoms in the ring and has two heteroatoms in the ring.

40. The method according to claim 39 wherein the heteroatoms of the cyclic donor molecule structure are located symmetrically on each side of the depicted carbonyl group.
